(19) United States
(12) Patent Application Publication
(10) Pub. No.: US 2012/0010603 A1
(43) Pub. Date: Jan. 12, 2012

(54) TISSUE OPTICAL CLEARING DEVICES FOR SUBSURFACE LIGHT-INDUCED PHASE-CHANGE AND METHOD OF USE

(75) Inventors: Thomas E. Milner, Austin, TX (US); Joseph Neev, Laguna Beach, CA (US)

(73) Assignee: Dermalucent, LLC, Austin, TX (US)

(21) Appl. No.: 13/158,086

(22) Filed: Jun. 10, 2011

Related U.S. Application Data
(63) Continuation-in-part of application No. 11/502,687, filed on Aug. 12, 2006.
(60) Provisional application No. 61/353,213, filed on Jun. 10, 2010.
(60) Provisional application No. 60/707,778, filed on Aug. 12, 2005.

(51) Int. Cl. A61N 5/067; A61B 18/18; A61N 5/06

(52) U.S. Cl. 606/13; 607/88; 607/89

(57) ABSTRACT

Tissue optical clearing devices for subsurface photodisruption and methods of use generally comprise an energy source in conjunction with mechanical optical clearing for the creation of high precision surface and subsurface photodisruption and/or photoablation.

### CROSS-REFERENCE TO RELATED APPLICATIONS

This application claims priority to U.S. Provisional Application Ser. No. 61/353,213, filed Jun. 10, 2010 and claims priority as a continuation-in-part to U.S. patent application Ser. No. 11/502,687, filed on Aug. 12, 2006, which claims priority to U.S. Provisional Application Ser. No. 60/707,778, filed on Aug. 12, 2005, all the aforementioned applications are herein incorporated by reference in their entirety.

### BACKGROUND

The invention generally relates to manipulating the optical properties of tissue for diagnostic and/or therapeutic advantage.

The specific morphology of human skin as well as other biological tissues gives rise to scattering of light. Skin may be the largest organ in the human body and composed of three distinct structures, as shown, for example, in FIG. 1. From the surface downward, they may be: 1) the epidermis; 2) dermis; and 3) subcutaneous fat (not shown). The epidermis may be the thinnest structure, varying in thickness from about 40 μm on the eyelids to about 1.6 mm on the palmar surface of the hand. The average epidermal thickness may be about 100 μm. The most superficial layer of the epidermis may be a dead outer layer, the stratum corneum, which may be responsible for the skin's chemical impermeability. Together, the stratum corneum and epidermis protect the human body from a variety of insults of physical, chemical, electrical, radiologic or microbiologic origin. The remainder of the epidermis may be a metabolically active, stratified squamous, cornifying epithelium generally populated by four types of cells: keratinocytes, melanocytes, Langerhans cells and Merkel cells in descending population frequency.

Keratinocytes form the bulk of the epidermis and undergo a specific form of cellular differentiation which may create the dead, superficial layers of the skin. Melanocytes, located in the deeper layers of the epidermis (basement membrane) may be capable of producing melanin which comprises the pigmentary system of the skin. Langerhans cells may serve an immunological function related to macrophages. Merkel cells may be receptors presumed to be involved in touch perception. The optical scattering properties of the epidermis can be characterized by the scattering coefficient (μₛ) and the anisotropy factor (g) or sometimes by the reduced scattering coefficient (μₛ′). The anisotropy factor is commonly represented by g and given by the mean of the cosine of the scattering angle, g=<cos(θ)>. In most tissues, light is predominantly forward scattered (θ is small) so that g can be close to 1. For example, Salomatina measured the reduced scattering coefficient of epidermis of human skin between 400 nm-1600 nm. Wan et al. reported scattering properties of the epidermis from the visible to the near infrared 800 nm. The reported reduced scattering coefficient of epidermis decreases as wavelength is increased from visible to infrared wavelengths. For example, Salomatina reports (FIG. 2a) that the reduced scattering coefficient of the epidermis decreases by more than two times as the wavelength increases from 500 nm to 800 nm. Salomatina et al. “Optical properties of normal and cancerous human skin in the visible and near-infrared spectral range”, Journal of Biomedical Optics 11(6), 064026 (November/December 2006). In epidermis, the anisotropy factor at visible wavelengths is approximately g=0.8 while values at infrared wavelengths is similar.

The dermis may be much thicker (1-2 mm) than the epidermis and may be subdivided into two compartments: 1) a thin zone immediately below the epidermis—the papillary dermis; and 2) a thick zone that extends from the base of the papillary dermis to the subcutaneous fat—the reticular dermis. The papillary dermis may be characterized by a network of thin (0.3-3 μm diameter) collagen fibers and elastic fibers (10-12 μm diameter), embedded in loose connective tissue and a highly developed microcirculation composed of arterioles, capillaries and venules. The reticular dermis may be composed predominantly of dense bundles of thick (10-40 μm diameter) collagen fibers that may be arranged primarily parallel to the skin's surface, interspersed among which may be coarse elastic fibers and fibroblasts embedded in an amorphous ground substance material containing water, electrolytes, plasma proteins and mucopolysaccharides. The latter consist of long-chain glycosaminoglycans which retain water in amounts up to about 1000× their own volume. The optical scattering properties of the dermis can be characterized by the scattering coefficient (μₛ) and the anisotropy factor (g) or sometimes by the reduced scattering coefficient (μₛ′). The anisotropy factor is commonly represented by g and given by the mean of the cosine of the scattering angle, g=<cos(θ)>. In most tissues, light is predominantly forward scattered (θ is small) so that g can be close to 1. For example, Salomatina measured the reduced scattering coefficient of dermis of human skin between 400-1600 nm. Wan et al. reported scattering properties of the dermis from the visible to the near infrared 800 nm. S. Wan, et al. “Analytical modeling for the optical properties of the skin in vitro and in vivo applications”, Photochem Photobiol, 34:493-499, 1981. The reduced scattering coefficient reported of dermis decreases as wavelength is increased from visible to infrared wavelengths. For example, Salomatina reports that the reduced scattering coefficient of the dermis decreases by about two times as the wavelength increases from 500 nm to 800 nm.

The scattering properties of tissue (e.g. human skin) substantially constrain the development of novel approaches for both light-based therapeutics and diagnostics. When light is incident on tissue (e.g., skin) the strong scattering redirects light in tissue so that light may not be easily focused to a targeted spatial region as in transparent media such as cornea. The strong optical scattering decreases the incident irradiance (E, W/cm²) as light propagates longer optical pathlengths in tissue—at longer optical pathlengths of light in a highly scattering tissue irradiance is decreased. In addition, the pulse duration (τ) may be lengthened with increased optical pathlength due to group velocity dispersion further decreasing the incident irradiance (E) in tissues. A number of optical interactions occur in materials (e.g., tissues) that scale with some power (p) of incident irradiance (E, W/cm²), E^{p}. For example, rate of temperature increase corresponding to single-photon or linear absorption scales with the first power (p=1) of the incident irradiance. Many multi-photon or non-linear interactions between light and materials are recognized in the art in which a particular type of interaction scales with a power of the incident irradiance (E′) with a power greater than unity (p>1). For example, plasma ablation using short pulses of light occurs when the irradiance exceeds a threshold value and has a power (p) dependence on irradiance that is greater than unity (p>0). In a given material (e.g., tissue) a variety of linear (single-photon) and non-linear (multi-photon) interactions exist and can occur simultaneously. The strong scattering of light in tissue severely limits the ability to achieve non-linear (multi-photon) interactions between light and tissue. Because magnitude of a non-linear (multi-photon) interaction scales with a power of the incident irradiance (E^{p}) where p greater than unity, decreased incident irradiance due to scattering substantially reduced magnitude of the non-linear interaction dropping below threshold at many depth position in the material (e.g., tissue). An important and notable exception is the cornea in the human eye. The cornea is a weak scattering tissue and allows light to propagate through the tissue with little scattering. The weak scattering properties of the cornea are utilized to achieve non-linear interactions between short-pulsed laser light to cut and remove corneal tissue with high precision. For example, high precision subsurface ablation using femtosecond laser pulses was reported in transparent biological materials such as cornea and applied successfully in intrastromal corneal refractive surgery (T. Juhasz et al., “Corneal refractive surgery with femtosecond lasers,” IEEE Journal on Selected Topics in Quantum Electronics 5(4), 902-910 (1999).

The present invention attempts to solve these problems, as well as others.

### SUMMARY OF THE INVENTION

Provided herein are systems, methods and compositions for achieving spatially confined phase-changes at depth in highly scattering tissues resulting from linear (single-photon) and non-linear (multi-photon) optical interactions. More specifically, methods and compositions are described that allow for increasing incident irradiance (E) of a pulse of light above relevant threshold(s) for a phase-change resulting from one or more linear (single-photon) and/or non-linear (multi-photon) interactions and target at least one of these optical interaction(s) at least at one position in a highly scattering tissue. The methods and compositions described herein to target a phase-change in a highly scattering tissue with pulses of light are relevant to the art of laser surgery. More specifically, the methods and compositions described herein are relevant to the laser surgical arts and allow targeting locations or structures in tissues for modification that include cutting, ablating, tearing, heating or generally any light-induced phase-change resulting from light absorption

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1 is a cross-section illustrating the morphology of human skin.

FIG. 2 is a block diagram of illustrating a process of events caused by mechanical force on tissue and resulting in desirable optical domain effects according to an embodiment of the disclosure.

FIG. 3A illustrates a light-based therapeutic embodiment of the disclosure without optically engineered human skin; FIG. 3B illustrates a light-based therapeutic embodiment of the disclosure with optically engineered human skin in which light scattering may be reduced and increased fluence may be delivered to the target chromophore; FIG. 3C is a schematic diagram demonstrating the mechanisms of plasma initiation by nanosecond and femtosecond lasers; and FIG. 3D is double-logarithmic map illustrating the five basic laser-tissue interactions.

FIG. 4A illustrates an example embodiment of an optical clearing device; FIG. 4B illustrates an example embodiment of an optical clearing device appressed to a tissue, wherein irradiation passes through two radiant filters; FIG. 4C illustrates an example embodiment of an optical clearing device with cooling, wherein the device is appressed to a tissue and a thermally-controlled fluid is flowing through the flow chamber, which is defined by the base, brim, and appressed skin; FIG. 4D illustrates an example embodiment of a pin of an optical clearing device contacting skin that is being irradiated, wherein the skin may transfer heat to a thermally-controlled fluid by one or more of the indicated pathways; and FIG. 4E illustrates an example embodiment of an optical clearing device with a fractional cooling system with a sapphire ball lens, wherein the skin may transfer heat to a thermally-controlled fluid by one or more of the indicated pathways.

FIG. 5 illustrates a structural embodiment of a treatment device comprised of an array of light emitting diodes, which may be positioned against the skin, resulting in the formation of a closed chamber between the LED array and skin.

FIG. 6A is an isometric drawing (posterior surface on the left) of an example embodiment of radiant filters in an optical clearing device which may used to control spatial and angular distribution of incident radiant energy into targeted tissue regions; FIG. 6B illustrates an example embodiment of an array of pins corresponding to the radiant filters, wherein the array defines an inner surface of the optical clearing device and may contact and/or apply a mechanical force on the skin; FIG. 6C illustrates an example embodiment of handpiece interface tabs of an optical clearing device which may provide a mechanical linkage between the device and the laser handpiece; and FIG. 6D illustrates an example embodiment of inlet and exit ports of an optical clearing device which may permit integration of vacuum and cooling systems with the device and tissue surface.

FIG. 7A is a schematic diagram of the one embodiment of the mechanical transducer; FIG. 7B is a schematic diagram of the second embodiment of the TOCD; FIG. 7C is a schematic diagram of the third embodiment of the TOCD; and FIG. 7D is a schematic diagram of observation of subsurface femtosecond ablation.

FIGS. 8A-8B are photographs of ex vivo rat skin with the TOCD 100 embodiment, where FIG. 8A is a back reflection image and FIG. 8B is a transmission image.

FIG. 9A is an image mosaic of histology images of subsurface femtosecond ablation using one TOCD embodiment with pulse energy of 26 uJ; and FIG. 9B is a close-up histological image of subsurface femtosecond ablation of circled area in FIG. 9A.

FIG. 10A is an image mosaic of histology images of subsurface femtosecond ablation using one TOCD embodiment with pulse energy of 40 uJ; and FIG. 10B is a close-up histological image of subsurface femtosecond ablation of circled area in FIG. 10A.

FIG. 11A is an image mosaic of histological images of subsurface femtosecond ablation using third TOCD at pulse energy of 52 uJ and the scale Bar of 1 mm; and FIG. 11B is a close-up histological image of subsurface femtosecond ablation of circled area in FIG. 11A with the scale Bar 100 um.

FIG. 12A is an image mosaic of histological images of subsurface femtosecond ablation using third TOCD at pulse energy of 69 uJ and the scale Bar of 1 mm; and FIG. 12B is a close-up histological image of subsurface femtosecond ablation of circled area in FIG. 12A with the scale Bar 100 um.

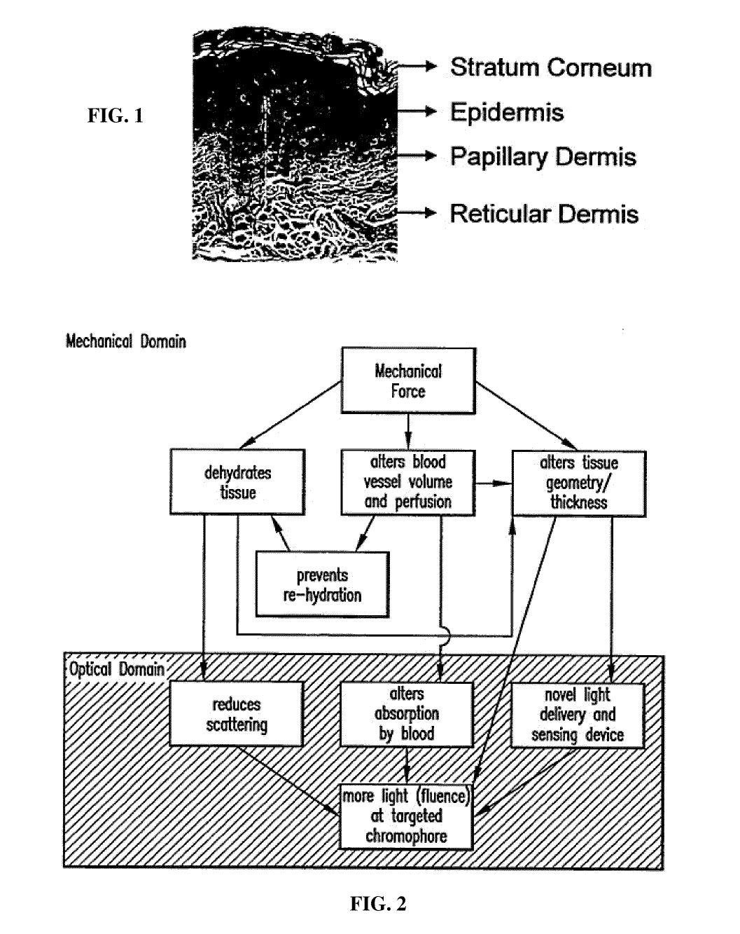

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Generally speaking, the mechanical optical clearing devices and methods of use comprise high precision subsurface photoablation that is generated using femtosecond or picosecond laser pulses. Femtosecond or picosecond pulses of light may be incident on scattering tissues, such as superficial rat skin or human sclera. The mechanical optical clearing devices allow for improved subsurface femtosecond photoablation in tissue. In this context, “photoablation” corresponds to a spatially localized phase-change induced by light and a sub-surface photoablation of the material has a phase change and is not immediately removed from the region of interaction. The mechanical optical clearing method may have a variety of applications, including, but not limited to cellulite (by cutting connective tissue connecting muscle and dermis), hair-removal, skin-tightening, wrinkle reduction, breaking down tattoo inks, adipose removal, or forming a protective barrier to sunlight.

In one embodiment, the mechanical optical clearing device includes a beam, wherein the beam is focused to a spot size of about 2 μm. Alternatively, the spot size may be between from 0.2 μm to 100 μm, as generally the irradiance is above a threshold. The threshold for larger spot sizes decreases the irradiance with the square of the spot size. Generally, spot sizes decrease as the wavelength increases. In one embodiment, the beam is scanned using a 2-dimensional motorized linear actuator at a speed of about 2 mm/sec. Alternatively, the beam is scanned speed should be variable from about 0.1 to 10,000 mm/s. In one embodiment, the beam focus location is below the air-epidermal interface and can be varied over a range of depths from 200 μm to 4000 um. Multi-photon optical microscopy or spectral-domain optical coherence tomography may be used to examine skin specimens before or following laser irradiation.

The mechanical optical clearing devices and methods include inducing photodisruption inside the skin without mechanical disruption of the overlying tissue. The mechanical optical clearing devices and methods allow rapid subsurface tissue modification (e.g. cutting) by the femtosecond laser, as further described below.

In one embodiment, light energy from a femtosecond laser produces high precision surface and subsurface photoablation in the skin. The femtosecond laser beam is capable of plasma induced photodisruption of various materials. Photodisruption is an irradiance dependent process and only occurs when the peak power of laser beam is above a threshold to transform or phase-change the material into plasma. The following plasma expansion causes permanent damage to the material and usually leaves a cavity at the expansion center. Generally speaking, the higher irradiance (intensity) produces a larger cavity and the cavity size obtained is about the size of the beam focus. Cavity size increases with pulse energy. Femtosecond laser photodisruption includes unique advantages over longer pulsed laser photodisruption. Femtosecond pulses are tens to a few hundred femtoseconds so that small pulse energy is required to reach damage threshold. The laser pulse transfers energy to the electrons through multiphoton absorption during irradiation. Absorption of femtosecond radiation is wavelength independent and does not require light wavelength match to absorption characteristics of the material. Femtosecond laser photodisruption only occurs at the focal volume due to nonlinear nature of the absorption. Subsurface photodisruption can be achieved in the materials that are transparent to femtosecond laser wavelength at low irradiance. Application of a tissue clearing device can allow femtosecond light to focus in a material so that irradiance increases above the threshold for photodisruption.

High precision subsurface femtosecond photodisruption may be achieved in transparent biological materials, such as cornea and corneal refractive surgery. Subsurface femtosecond photodisruption in turbid or highly scattering tissues may be done in tissue, such as in skin and sclera, as indicated below. Producing subsurface photodisruption in scattering tissues is more difficult than in transparent tissues. Photons deviate from straight trajectories due to non-uniformities of refractive index in scattering tissues and do not contribute to nonlinear absorption at a target focal volume. As a result without tissue clearing, subsurface photodisruption occurs only when femtosecond pulses are focused superficially in scattering tissues. The mechanical optical clearing device produces subsurface cavities (˜10 μm diameter) up to 100 μm beneath the surface of various skin samples. Increasing pulse energy to overcome scattering losses can help to produce deeper cavities, but a different damage mechanism is triggered when the beam is focused more than 100 μm beneath the surface. Fan-like filaments originating from the laser focus are produced by the mechanical optical clearing device instead of well defined subsurface cavities due to self focusing of high energy laser beam. For the mechanical optical clearing device, the relation between penetration depth and laser wavelength for femtosecond pulses with a wavelength range from 1100 nm to 2200 nm include much less scattering then those with a wavelength around 775 nm in human sclera and human skin specimen, respectively. Although absorption by water increases dramatically in the above wavelength range, subsurface photodisruption may be produced in scattering human sclera at a depth of 500 μm using 1700 nm wavelength femtosecond pulses, whereas 775 nm femtosecond pulses may obtain a penetration of 250 μm.

In an attempt to provide deeper delivery of focused femtosecond beam with reduced scattering, potentially producing deeper subsurface photodisruption in scattering tissues, tissue optical clearing using chemical agents may be utilized and provide substantial effects in reducing scattering properties of tissue specimens. Subsurface photodisruption is observed 500 μm below the surface of human sclera samples for both 1060 nm and 775 nm wavelength femtosecond lasers in the case that chemical clearing agent, Hypaque, was applied. Enhancement of femtosecond pulse delivery in ex vivio porcine skin tissue is demonstrated after application of glycerol, another type of chemical clearing agent. And depth enhancement of femtosecond pulse delivery in ex vivo porcine skin was demonstrated after application of glycerol, a chemical clearing agent. Ablation depths in porcine skin may be increased by 200 to 400 μm for an NA=0.1 focusing lens and a maximum 1.2 mm depth of femtosecond ablation as measured using ultrasound.

Tissue optical clearing devices and methods using a mechanical force enhance light penetration in scattering tissues. In one embodiment, tissue optical clearing using mechanical force is applied with Tissue Optical Clearing Devices (TOCD) to provide mechanical force or vacuum-related compression to displace interstitial or intracellular water in tissue, therefore increase collagen concentration, reduce refractive index mismatch, and provide enhancement of light penetration. The penetration depth for the TOCD may be between 1 and 1400 μm. Optical penetration depth is deepest in the wavelength region from 800 to 1100 nm. Light with a wavelength in this region is preferable for applications that require deep skin penetration

Demonstration of TOCD on ex vivo porcine skin showed clear evidence of penetration enhancement for visible and near infrared wavelength, as indicated below. There are unique advantages in favor of TOCD over chemical methods. The TOCD method is a less invasive procedure, since the TOCD method does not require to break the barrier function of the epidermis layer and apply chemical agents. TOCD provides longer operation time for the following experiments or surgeries and is easy for tissue to recover from dehydrated state.

Skin

Skin may act as a highly scattering medium for visible to near-infrared wavelengths due to its complex and inhomogeneous morphological structure. Light scattering in biological tissues may be caused primarily by variation in polarizability, which may be characterized by variations of the optical index of refraction n. This mismatch may be largely a function of shape, size and distribution of tissue constituents such as collagen (70% of dry weight of dermis), lipids, water, cells and their organelles, which all have slightly different indices of refraction as shown in Table 1. While the variations of optical index of refraction V n give rise to scattering, strength and direction of scattered light depends on size and shape of the scatterers. Light scattering from structures with size much smaller than the wavelength λ of incident radiation is governed by Rayleigh theory. Alternatively, light scattering from structures with size comparable to the wavelength λ of incident radiation is governed by Mie theory. In the epidermis, melanin granules and keratinocyte and melanocyte nuclei provide large ∇n that give rise to Rayleigh and Mie scattering. In the papillary and reticular dermis, Mie scattering may be due to ∇n at the interface between high-index collagen fibers and surrounding low-index extracellular fluid, ground substance and cytoplasm.

TABLE 1
Optical index of refraction of different tissue constituents

| Tissue/Cell Component | Refractive Index |
|---|---|
| water | 1.33 |
| collagen | 1.43 |
| hydrated collagen | 1.53 |
| dehydrated melanin | 1.7 |
| stratum corneum | 1.55 |
| adipose tissue | 1.46 |
| extracellular fluid | 1.35 |
| cytoplasm | 1.37 |
| nucleus | 1.39 |
| mitochondria | 1.42 |

There may be a natural gradient of water content as a function of depth throughout skin. At the stratum corneum, water content may depend on atmospheric humidity and may be as low as about 15% and increase with depth. At a depth of about 35 μm the water content of epidermis and dermis may reach about 70%. Thus water, with its lower optical index of refraction contributes significantly to the index of refraction mismatch V n giving rise to light scattering. The penetration depth of light may be limited due to attenuation, which results from light scattering and light absorption within biological tissue. The effective penetration depth of light (Lp) in absorbing and scattering tissues can be approximated as:

$${L}_{p} = \frac{1}{\sqrt{3 {α}_{a} ( {α}_{a} + ( 1 - g ) {α}_{s} )}} , \qquad ( 1 )$$

where αₛ is the scattering coefficient, which is the inverse of scattering length, αₐ is the absorption coefficient, and g=1 and corresponds to the case of purely forward scattering, while g=−1, which corresponds to purely backward scattering. For skin, typical values of g are in the range of 0.7-0.95, and vary with wavelength.

Significant tissue chromophores that absorb laser energy in skin may include water, melanin and hemoglobin in blood. In light-based therapeutics, successful treatment outcome may require a temperature increase by absorption of incoming photons. Specifically, successful treatment outcome may depend on a desired temperature increase in selected tissue regions resulting in destruction of targeted chromophores, while maintaining temperature below the threshold for destruction in non-targeted tissue regions, as shown in FIG. 2.

Temperature increase ΔT (r, z) in tissue at position (r, z) may be given by:

$$Δ T = \frac{{μ}_{a} \cdot Φ}{ρ \cdot c} , \qquad ( 2 )$$

and may be dependent on tissue absorption coefficient (μₐ), optical fluence Φ and tissue heat capacity ρ·c. In tissue regions targeted for destruction, desired temperature increase may be obtained by either increasing μₐ or Φ or decreasing ρ·c or some combination thereof. For example, tissue absorption coefficient μₐ may be increased at a selected position by increasing chromophore density at that position. Fluence Φ may be increased at a given position (rₒ, zₒ) by decreasing tissue scattering at overlying positions (z<zₒ). The tissue heat capacity ρ·c may be decreased by reducing water concentration. Likewise, in tissue regions not targeted for destruction, temperature increase may be lessened by either decreasing μₐ or Φ or increasing ρ·c or some combination thereof.

TABLE 2
Pathologic conditions amenable to light-based therapies

| Vascular Lesions | Pigmented Lesions | Other Skin Pathologies |
|---|---|---|
| Hirsutism | Cellulite | Skin cancers PDT |
| Port Wine Stains | Lentigo | Acne vulgaris |
| (PWS) | | |
| Hemangiomas | Nevus of Ota | Acne scars |
| Telangiectasis | Nevus of Ito | Hypertrophic scars |
| Angiomas | Blue nevus | Rhytides |
| Adenoma sebaceum | Ephelides | Hypertrichosis |
| Angiokeratomas | Becker's nevi | Hidradenitis suppurative |
| Venous lakes | Hairy nevi | Pseudo-folliculitis barbae |
| Spider veins | Epidermal melanosis | Tattoos |
| Rosacea | Nevus spilus | Chrysiasis |
| Poikloderma of Civatte | Hyper-pigmentation | Adipose contouring |
| | Skin Laxity | Adipose removal |
| | | Adipose reduction |

Light-based diagnostics and/or therapeutics may benefit from reduced human skin light scattering by two different pathways: 1) increased effective fluence that reaches the target chromophore; and 2) reduced overall backscattered light from the dermis reaching non-targeted chromophores such as melanin in the epidermis which increases the latter's threshold for injury.

This may be particularly important in patients with darker skin types where it may not be possible to target chromophores with sufficiently high therapeutic dosage due to non-specific epidermal damage. Taken together, reduced light scattering and re-distribution of tissue chromophores may not only make light-based therapeutic procedures more effective, but also safer. A review of some pathologic conditions amenable to light-based therapies that may benefit from optically engineered human skin is provided in Table 2.

Port wine stain (PWS) may be a congenital, progressive vascular malformation of skin that occurs in an estimated 4 children per 1,000 live births. Approximately 1,500,000 individuals in the United States and thirty-two million people worldwide have PWS birthmarks. Since most of the malformations occur on the face, PWS may be a clinically significant problem in the majority of patients. To some extent, PWS may be considered a cosmetic problem, but it may also be regarded as a disease with potentially devastating psychological and physiological complications. Personality development may be adversely influenced in virtually all patients by the negative reaction of others to a “marked” person. Detailed studies have documented lower self-esteem in such patients and problems with interpersonal relationships. Studies have indicated a high level of psychological morbidity in PWS patients resulting from feelings of stigmatization that may be frequently concealed in casual social interactions. In childhood, PWS may be flat red macules, but lesions tend to darken progressively to purple, and by middle age, often become raised as a result of the development of vascular nodules. Hypertrophy of underlying soft tissue, which occurs in approximately two-thirds of lesions, further disfigures the facial features of many patients.

Histopathological studies of PWS show a normal epidermis overlying an abnormal plexus of dilated blood vessels located in the dermis. PWS blood vessel diameter (30-300 μm) and depth distribution (200-750 μm) vary on an individual patient basis and even between different areas on the same patient. Pulsed dye laser (PDL) treatment, which may selectively destroy dermal blood vessels, typically results in a variable and unpredictable degree of blanching. If the ultimate standard required may be complete blanching of the lesion, the average success rate may be below 10%, even after undergoing multiple PDL treatments. Without limiting the disclosure to any particular theory or mechanism of action, this may occur because of the inability to deliver an adequate effective light fluence to the targeted PWS blood vessels.

Methods and devices of the present disclosure may reduce light scattering and redistribute tissue structures regions or chromophores in optically engineered human skin. In some embodiments, mechanical optical clearing can move some tissue structures relative to the incoming laser beam and reduce the product of tissue scattering coefficient (μₛ) X_{d} (tissue depth). In some embodiments, methods and devices of the disclosure may increase the therapeutic success rate, improve safety, and/or decrease the number of repeat treatments as follows: 1. Reduced light scattering may permit safe and effective laser treatment of PWS by increasing the threshold for epidermal damage. The best clinical results in patients with PWS undergoing laser therapy may be obtained when the ratio of heat generated in blood vessels to that in the epidermis may be highest. 2. For patients with darker skin types, previous methods did not permit treating lesions with a sufficiently high therapeutic light dosage due to epidermal damage. Reduced light scattering and localized reduction of melanin concentration due to skin stretching may expand the population of patients expected to benefit from laser therapy by increasing the threshold for epidermal damage. 3. A principal reason for poor clinical results seen after laser therapy of patients with PWS may be insufficient heat generation within large blood vessels. Multiple treatments at low light dosages will not achieve and sustain the critical temperature necessary to destroy irreversibly large blood vessels, regardless of the number of treatments performed. Optically cleared skin may permit the use of higher incident light dosages to produce higher intravascular temperatures, over longer periods of time without producing permanent complications such as hypertrophic scarring, changes in skin pigmentation, atrophy, or induration.

Without being limited to any particular mechanism of action, reduction of light scattering may be achieved by three mechanisms, which include local tissue dehydration, index of refraction matching and structural modification of proteins such as collagen. Local tissue dehydration may cause tissue shrinkage and bring individual scattering centers closer together, thereby reducing light scattering in highly turbid skin.

Optical skin clearing induced by delivery of hyper-osmotic agents such as glycerol or dextrose may be hindered since these agents typically diffuse poorly across the natural skin barrier formed by the stratum corneum. Furthermore, modification of the barrier by thermal, chemical or mechanical means requires additional time beyond diffusional mass transport. Consequently, maximum optical skin clearing has been observed, in some cases, at approximately 60 to 360 minutes after the application of the optical skin clearing agent.

Mass transport of interstitial water resulting in a re-distribution of this abundant tissue chromophore may be induced by osmotic stress. However, more efficient may be mechanical displacement and re-distribution of interstitial tissue water.

The present disclosure provides, in some embodiments, a device for controlled local (fractional) tissue dehydration and/or local skin compression and stretching resulting in redistribution of the most significant tissue chromophores including water, blood and melanin.

Without being limited to any particular mechanism of action, specific example embodiments of the disclosure may involve applying at least one mechanical force to a tissue (e.g., skin) to increase light transport to targeted chromophores and/or increase or decrease selected chromophore concentration in tissue. Application of a mechanical force may move intracellular and interstitial water out of a targeted tissue volume causing spatial redistribution of one or more scatterers and thereby reduce light scattering. Application of a mechanical force may increase or decrease blood volume fraction and perfusion and/or may modulate one or more natural mechanisms for maintaining tissue hydration. Application of a mechanical force may increase or decrease tissue volume or thickness.

Example embodiments of this disclosure may include a feedback device that provides a distribution of mechanical forces to skin for improved laser treatment of a variety of conditions, e.g., hair removal, blood vessel coagulation, skin rejuvenation, adipose contouring, adipose reduction, and adipose removal. An embodiment of a method of the disclosure applied to skin may comprise applying a lifting force (e.g., hypobaric pressure) that positions a tissue volume within a control volume of a device having one or more surfaces for applying a mechanical force to the tissue. A method of the disclosure may further comprise injecting light into the control volume and light sensors that provide a feedback signal to the mechanical force transducers. Using the feedback signal, the mechanical force transducers may be configured to provide optimal light fluence at the targeted chromophores.

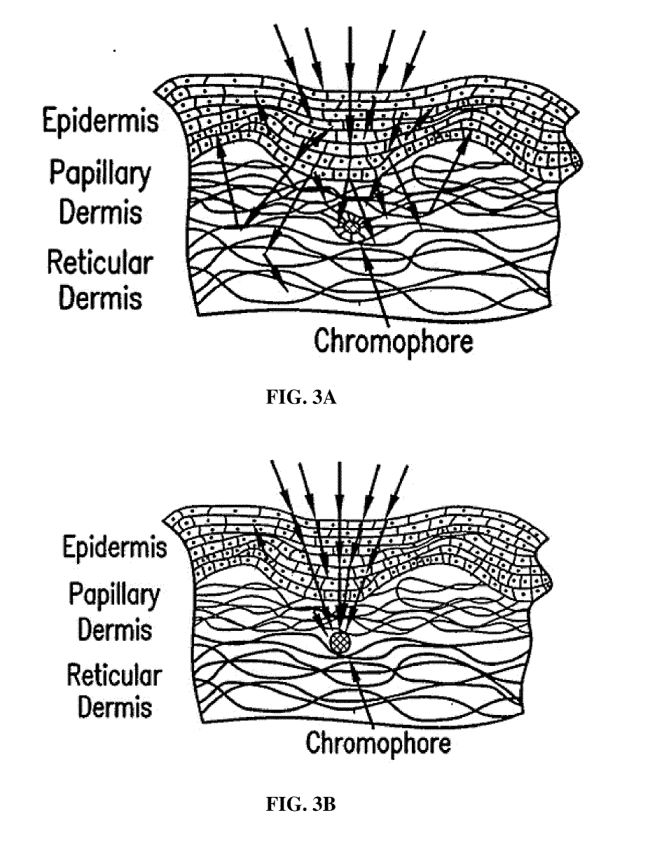

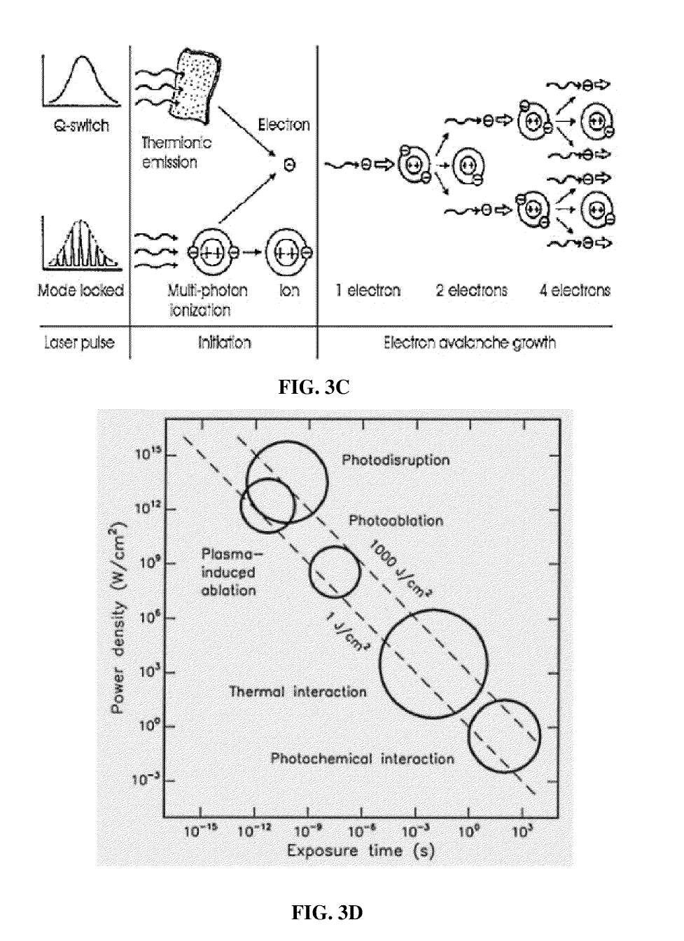

Techniques and devices of the disclosure may increase the probability of chromophore damage by increasing light fluence at the targeted chromophore. The device may accomplish this task by redistributing chromophores such as intracellular and interstitial water, blood and melanin, by decreasing the physical thickness of tissue, and by employing a light delivery and optionally a sensing methodology. Methods and devices of the disclosure may improve both optically based therapeutic and diagnostic procedures. The block diagram in FIGS. 3A-3B illustrates the events, according to some embodiments, associated with applying mechanical force on tissue and resulting in desirable optical domain effects.

There may be numerous functional elements that may be potentially embodied with different structure in devices of the disclosure. Some non-limiting examples of these are listed in Table 3.

TABLE 3
Functional Elements of the Disclosure

| Functional | | | | | |
|---|---|---|---|---|---|
| Elements | Structure | | | | |
| Radiant | Laser | Radio | Microwave | X-ray | Incoherent |
| Source | | Frequency | | | light source |
| | | | | | (e.g., LED or |
| | | | | | flashlamp |
| | | | | | source) |
| Mechanical | Pins | Vacuum | Clamp | Rolling | |
| Transducer | | | | Cylinder w/ | |
| | | | | Pins | |
| Radiant Filters | Mask | Conventional | Fresnel Lens | Holographic | Hybrid Lens |
| | | Lens | | Lens | |
| Cooling | Conduction in | Convective | Convective | Evaporative | |
| | Pins | Liquid (e.g., | Gas against | Liquid | |
| | | against pins, | (e.g., against | against (e.g., | |
| | | tissue, or | pins, tissue, | against pins, | |
| | | base) | or base) | tissue, or | |
| | | | | base) | |
| Feedback of | Optical | Ultrasonic | Mechanical | Electrical | |
| Optical | Feedback | Feedback | Feedback | Feedback | |
| Properties to | | | | | |
| Source | | | | | |
| Tissue | Vacuum | Clamp | | | |
| Position and | Device | | | | |
| Hold | | | | | |
| Tissue and | Melanin | Hemoglobin | Water | Dye | Lipid or fat |
| Chromophores | | | | | |

According to some embodiments of the disclosure, a system may comprise synergistic functional elements and structural embodiment including, without limitation, (1) a radiant source, (2) a mechanical transducer, (3) a tissue position and hold mechanism, (4) a radiant filter, (5) a cooling system, (6) a feedback system between a tissue and a radiant source, and (7) tissue to be treated.

(1) Radiant Source

The radiant source generates electromagnetic energy that will ultimately be delivered to targeted tissue chromophore(s). This functional element may be structurally embodied as a laser, incoherent (flashlamp), light emitting diode (LED), superluminescent diodes (SLD), radio frequency, microwave, or x-ray source.

For example, a laser source may, in some embodiments, provide a dosimetry (e.g., wavelength, fluence, pulse duration, and/or spot size) that may be selected to target specific structures and/or material compositions in a tissue. In some embodiments, a tissue may be targeted by using the principles of selective photothermolysis, photoablation, or photodisruption.

The laser methods, include by way of example and not limitation, a femto-second laser, an excimer laser, a fiber laser chirped pulsed amplifiers, or other laser sources and combinations. Femtosecond lasers are lasers that emit optical pulses with aduration well below 1 ps (ultrashort pulses), i.e., in the domain of femtoseconds (1 fs=10⁻¹⁵ s). Femtosecond lasers may include Bulk Lasers, Fiber Lasers, Dye Lasers, Semiconductor Lasers, titanium-sapphire lasers, and the like. Passively mode-locked solid-state bulk lasers can emit high-quality ultrashort pulses with typical durations between 30 fs and 30 ps. Various diode-pumped lasers, e.g. based on neodymium-doped or ytterbium-doped gain media, operate in this regime, with typical average output powers between 100 mW and 1 W. Titanium-sapphire lasers with advanced dispersion compensation are suitable for pulse durations below 10 fs and down to approximately 5 fs. The pulse repetition rate is between about 50 MHz and 500 MHz, even though there are low repetition rate versions with a few megahertz for higher pulse energies, and also miniature lasers with tens of gigahertz.

Various types of ultrafast fiber lasers, which are also in most cases passively mode-locked, typically offer pulse durations between about 50 and 500 fs, repetition rates between about 10 and 100 MHz, and average powers of a few milliwatts. Substantially higher average powers and pulse energies are possible, e.g. with stretched-pulse fiber lasers or with similar lasers, or in combination with a fiber amplifier. Dye lasers include a gain bandwidth that allows for pulse durations of the order of 10 fs, and different laser dyes are suitable for emission at various wavelengths, often in the visible spectral range. Some mode-locked diode lasers can generate pulses with femtosecond durations. Directly at the laser output, the pulses durations are usually at least several hundred femtoseconds, but with external pulse compression, much shorter pulse durations can be achieved. Vertical external-cavity surface-emitting lasers (VECSELs) can be passively mode-lock, which can deliver a combination of short pulse durations, high pulse repetition rates, and sometimes high average output power. Other types of femtosecond lasers are color center lasers and free electron lasers, where the latter can be made to emit femtosecond pulses even in the form of X-rays.

Alternatively, the pulses may include beamlets. Scaling of the optical efficiency with number of foci produce decorrelated beamlets that contain all of the available laser power. A common method of producing multiple foci in multiphoton microscopes relies on placing a microlens array in the beam to produce a series of beamlets. These beamlets are then appropriately relayed to the entrance pupil of the objective and scanned with galvanometric scanners or with a rotating lenslet array. Creating beamlets with ultrashort laser pulses (1 ps) need to have the interference between the out-of-focus tails of the pulses be eliminated or reduced if the pulses arrive at different times. The beamlets must be parallel so they can be properly relayed to the entrance aperture of the microscope objective. One method relies on the propagation of the beamlets through different lengths of material, as described in D. N. Fittinghoff, C. B. Schaffer, E. Mazur, and J. Squier, “Time-decorrelated multifocal micromachining and trapping,” IEEE J. Sel. Top. Quantum Electron. 7, 559-566 (2001). Alternatively, a Yb:KGd(WO₄)₂ oscillator design that generates six beams of temporally delayed, 253 fs, 11 nJ pulses. Sheetz, et al. “Advancing multifocal nonlinear microscopy: development and application of a novel multibeam Yb:KGd(WO₄)₂ oscillator”, 16 Optics Express 22 (2008).

When spatial power densities are higher than 10¹¹ W/cm₂, plasma-induced ablation becomes a dominant type of interaction. To achieve such a high power density, laser pulse duration is usually on the order of tens of nanoseconds to tens of femtoseconds. Initially, free electrons are generated by high intensity laser irradiation. Those free electrons accelerate by absorbing photons and then produce additional free electrons through impact ionization of nearby atoms. When the time of this process is much shorter than the laser pulse duration, multiple collisions can occur and cause so-called avalanche ionization. When a sufficient number of electrons are produced, a plasma is formed. Subsequently, plasma expansion creates high pressure gradients and shockwave generation. Shockwave propagation and subsequent vapor expansion can cause permanent damage and tissue ablation. The initiation of plasma generation depends on laser pulse duration. Nanosecond pulses have a completely different initiation mechanism than femtosecond pulses, as shown in FIG. 3C.

As shown in FIG. 3C, nanosecond or picosecond lasers (such as Q-switch laser) utilize a thermionic emission mechanism (thermal ionization) to produce seed electrons. The tissue is heated to extremely high temperatures (larger than thousands of Kelvin) so that free electrons are released. The plasma formation threshold depends on the local linear absorption coefficient of the tissue. Because impurity electrons are the main source of initial seed electrons, a pulse-to-pulse variation of the plasma threshold exists because of the probabilistic nature of free electron generation. Fluctuations in the number of seed electrons in the focal volume strongly affect the plasma generation threshold. With femtosecond pulses, initial seed electrons are produced by multiphoton absorption. Coherent absorption of several photons by one bound electron is ensured by the extremely high photon density of a femtosecond pulse. For femtosecond pulses, threshold of plasma generation is therefore deterministic and independent of impurity electrons. In femtosecond pulses, linear absorption coefficient of the tissue is found to have a weak effect on the plasma generation threshold

Because of the short duration of femtosecond pulses, the plasma generation threshold of power density is higher than that of nanosecond pulses, while the threshold energy density is lower. Less pulse energy is needed to produce femtosecond induced plasma. Therefore, plasma energy and plasma temperature are usually higher in nanosecond photoablation. Because the energy transfer time from free electrons to ions by collisions is on the order of several picoseconds (1 ps=1000 fs) and is much longer than the duration of a femtosecond pulse, only a small fraction of incident laser energy can be transferred to heat the ions and produces shock waves at the end of a femtosecond pulse. Amplitude of shock wave pressure for femtosecond pulses is thus smaller than that observed in nanosecond laser related plasma-induced ablation. Therefore, plasma-induced ablation achieved by nanosecond pulses is often accompanied by more collateral damage than that for femtosecond pulses.

At higher pulse energies, large shockwave pressures are produced and propagate outward. As a result, collateral damage becomes more significant for both nanosecond and femtosecond pulses. Because the effect of rupturing becomes more evident in adjacent regions of the irradiated tissue, the interaction is thus defined as photodisruption instead. As observed in FIG. 3D, a clear separation does not exist in power density or pulse duration between plasma-induced ablation and photodisruption. Photochemical, photothermal, photoablation, plasma-induced ablation, and photodisruption is illustrated in FIG. 3D. The distinction between plasma-induced ablation and photodisruption can also depend on irradiated tissue type.

(2) Mechanical Transducer

A mechanical transducer may apply force to the tissue that alters the tissue's mechanical state (e.g., thickness, stress, and strain) and optical properties. The tissue scattering coefficient (μₛ) may be modified by displacement of water and may result in increased density of scatterers. The tissue absorption coefficient may be modified by displacement/concentration alteration of chromophores. Additionally, tissue compression and changing of tissue thickness may improve radiant throughput by reducing the path length to a target in the tissue. Tissue compression may be fractional in some embodiments. For example, compression may occur at discrete points that constitute less than the total area enclosed by the brim.

In some embodiments, a mechanical transducer may include an array of pins that contact target tissue. Alternatively, a mechanical transducer may be embodied as a clamp, where two surfaces (pinned or flat) may be forced together with tissue in-between. In this embodiment, a tissue volume may be positioned between two surfaces either of which contains mechanical transducers such as a pin array. Application of a clamping force (e.g., spring, screw, piston, pneumatic) forces the pins into the tissue, displacing water and modifying tissue optical properties. Alternatively, the mechanical transducer may be an indenter. The indenter may be three-sided pyramid, four-sided pyramid, a cone, a cube corner, a sphere, and the like.

In some embodiments, a mechanical transducer may include one or more sub-components selected from the group consisting of an array of pins, a brim, a base, an inlet and exit port and tubing, and a handpiece interface tab. In some embodiments, apart from the inlet and exit port tubing, the device may be constructed in a single manufacturing process using a single material. The device may be constructed of a variety of materials transparent to incident radiant energy such as glass, clear polymer, or similar optical material so radiant energy may be applied to the tissue while the device may be in position and functioning. A thermally conductive material, such as sapphire may be desirable to aid in the tissue cooling function. Inlet and exit port tubing may consist of commercially manufactured polymer hose. The device may be manufactured through thermoplastic molding or machining.

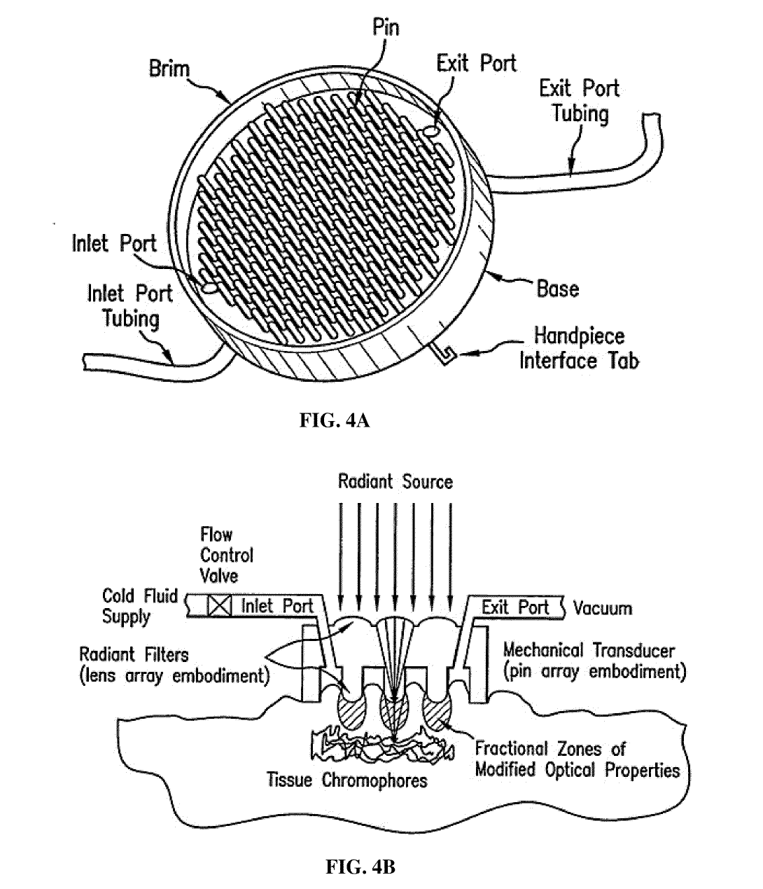

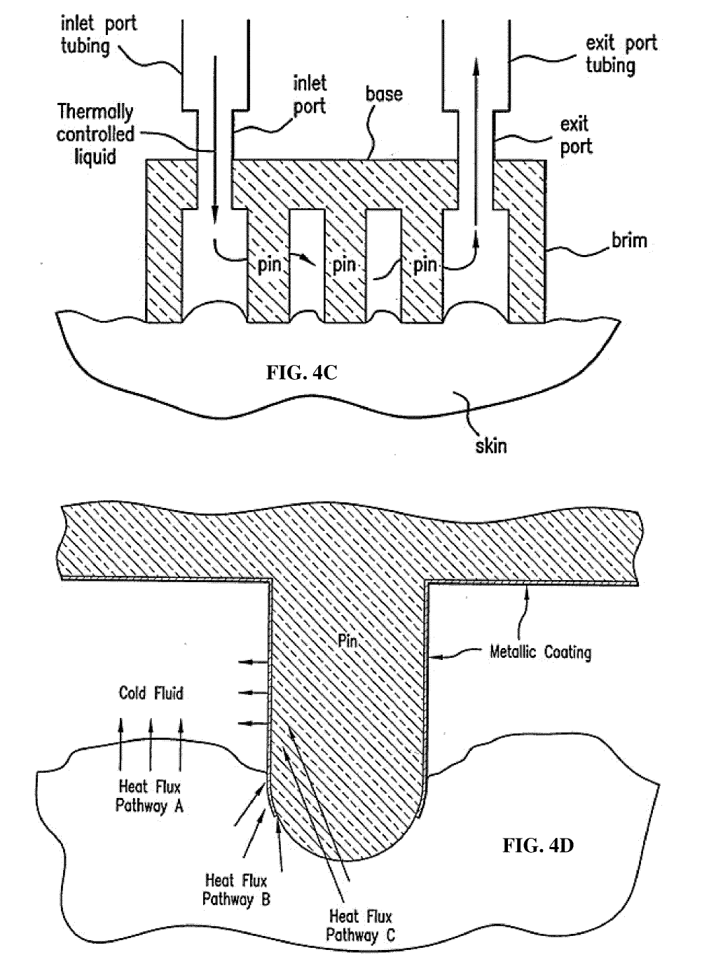

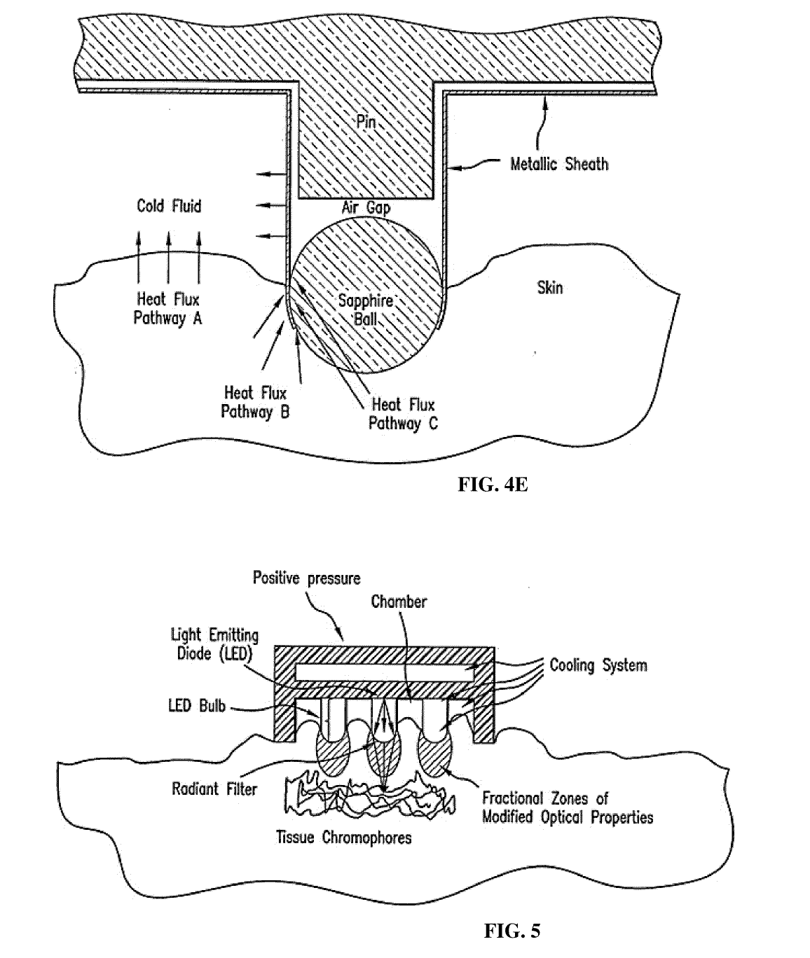

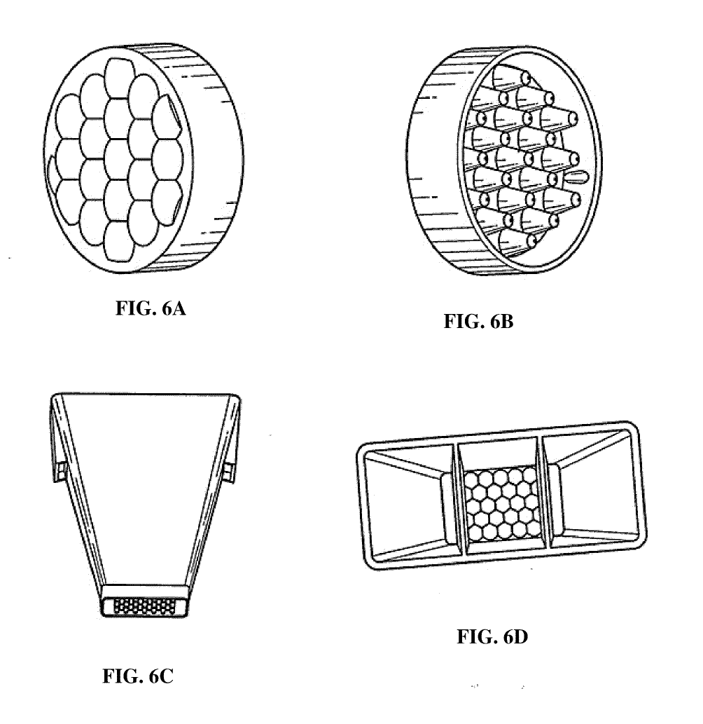

An example device of the disclosure may include a clear plastic resin, may be circular in shape, and may have a diameter of about 1.5 cm. The device shown in FIG. 4A features an outer brim, which acts as a seal once the device is placed onto the skin surface.

The mechanical transducer in combination of the vacuum may apply a pressure to the tissue or contact surface area. The pressure may range between about 0.01 mPa to about 100 mPa. The applied pressure may be altered depending on the tensile strength or tautness of the tissue.

(a) Array of Pins

Inside, facing the skin surface may be an array of pins, which may be constructed of a variety of optically transparent materials such as glass, clear polymer, or similar optical material so radiant energy may be applied to the tissue while the device is in position. While pins may be generally firm or rigid, in some embodiments, an array of pins may include at least one pin that may be resilient. An array of pins may be manufactured using known procedures including thermoplastic molding or machining.

Pin design parameters may include pin shape (e.g., flat, rounded), diameter, length, packing density, tip shape, arrangement, and number. Pins may be shaped as cylindrical rods, as shown in FIGS. 4A-4E, or with many other cross-sectional shapes (e.g., triangular, square, radial, or spiral). Pin diameter may be selected according to flux of water transport in response to tissue compression. Pin diameters on the order of 0.5 to 1.0 mm may provide fast (1-5 s) response of tissue optical property change. Smaller pin diameters may give faster water transport and more rapid change in tissue optical properties. However, the artisan of ordinary skill may, according to some embodiments, balance small pin diameter against a reduced applied pressure corresponding to risk of tissue penetration or puncture. In some embodiments, pin length may be sufficiently large to prevent skin contact with the device base and possible blockage of the inlet or exit port. Pin length of about 2-3 mm may be sufficient for a pin packing density greater than 20%. Density in this respect refers to the cross-sectional area of the pins at their tips as a fraction of the area defined by the anterior perimeter of the brim. A pin cross section for density calculation may also be at the base or midpoint. Pins on a single device may have a uniform length or from 2 to Q different lengths, where Q is the number of pins on the device.

Modification of local tissue optical properties may be more effective and rapid with low pin packing density, but the fractional area of compressed tissue may be diminished. Pin packing densities of 20-50% have been implemented successfully on some example devices. Pin tip shape (e.g., hemispherical, flat, conical) may simultaneously affect the stress profile in tissue and serve as a radiant filter. A hemispherical-shaped pin tip may distribute stress more uniformly in tissue, provide enhanced and rapid alteration of tissue optical properties, and simultaneously act as a lens embodiment of a radiant filter. A sapphire ball lens may be used to form a hemispherical-shaped pin tip that additionally provides a high conductivity path for thermal energy removal from the tissue just beneath the pin. A flat or planar-shaped pin tip may induce a ring of high tissue stress along the circumference of the pin tip, and may not induce a lensing effect. Pins may be arranged in many lattice geometries including, without limitation, uniformly (e.g., in a Cartesian grid, a checker-board pattern, a hexagonal pattern), haphazardly, or randomly distributed. The total number of pins, effective pin size, and pin packing density may determine the total size of the pin array. The pin array may be large enough to accommodate the entire cross-sectional area of the incident radiant source, and small enough to form a seal (e.g., hypobaric or pneumatic) with a curved tissue surface such as external facial structures. A pin array diameter on the order of 1-2 cm may be appropriate in some embodiments.

(b) Brim

A brim of a mechanical transducer may be the lip along the periphery of the device which may form an airtight or vacuum seal with the tissue surface. A brim may completely surround and enclose an array of pins. The shape of the brim may be coupled with the shape of the pin array. A brim may be laterally offset from the edge of the pin array by approximately 1-2 mm, e.g., to ensure an airtight seal. The length and width of a brim may be approximately the same as that of the pins (e.g., 1 mm). Shape of the brim tip that interfaces with skin may be circular, according to some embodiments, since this shape may conform with the tissue quickly and seal effectively.

(c) Base

A base of a mechanical transducer may be, in some embodiments, a flat plate which supports both the pins and the brim. The base may also provide a support structure for radiant filters. According to some embodiments, a base may be at least 2-3 mm thick in order to provide structural strength and rigidity of the device. In other embodiments, a base may be made of, for example, a flexible, resilient, and/or elastomeric material. A flexible base may permit, for example, an adjoining array of pins to better adopt the contours of a surface (e.g., tissue) with which it is contacted.

(d) Inlet and Exit Port and Tubing

Inlet and exit ports may permit the flow of fluid into and exiting the chamber (which may be formed when the device is applied to tissue). An inlet may include a single aperture or an array of apertures in the base. Similarly, an exit port may include a single aperture or an array of apertures in the base. In addition, port holes may be surrounded by a connector (e.g., a Luer fitting) extending about 3-5 mm from the posterior surface of the device base. Port tubing may be fastened to the device by compression into the port holes, or by expansion around Luer fittings. Inlet and exit port tubing may include, without limitation, commercially manufactured polymer hose.

(e) Handpiece Interface Tab

A handpiece interface tab may provide a mechanical linkage between a mechanical transducer device and a radiant source. For example, a handpiece interface tab may be structurally embodied as a tab with a flanged or curved tip. A user may press (or twist) a device against a radiant source handpiece, and the flanged or curved tip may be locked into a handpiece receptacle. When desired, a user may remove the device from the handpiece by gently forcing or twisting them apart.

(3) Tissue Position and Hold Mechanism

A tissue position and hold mechanism may facilitate alignment of the mechanical transducer element against the tissue and formation and/or maintenance of an airtight seal if a pneumatic embodiment is employed. A primary embodiment for the tissue position and hold mechanism may be a hypobaric vacuum chamber. In this embodiment an applied hypobaric pressure determines the vertical position of the tissue with respect to the mechanical transducers (e.g., array of pins). The tissue position and hold function may be structurally embodied with a pin array surface and/or vacuum device.

A pneumatic device may aid in the mechanical transduction process by forcing the tissue against the pins while simultaneously pulling tissue into the volume surrounding each pin (i.e., control volume). The increased tissue volume surrounding the pins (expanded tissue regions) provides additional storage capacity for water and blood displaced from under the pins. A pneumatic device may include a vacuum pump and a hose connecting the pump to the pin array device (e.g., exit port).

(4) Radiant Filters

The radiant filters may be utilized to control spatial and angular distribution of incident radiant energy into targeted tissue regions. The radiant filters and their spatial arrangement may be engineered specifically for each treatment application. For example, radiant filters may be used to direct radiant energy to mechanically compressed regions for hair removal, skin rejuvenation or adipose reduction treatments because scattering coefficient may be reduced (optimized) and water concentration may be reduced (optimized) in these regions. Radiant energy may be directed into the expanded tissue regions for blood vessel coagulation treatments because chromophore density may be highest (optimized) in these regions. The radiant filter function may provide an important accessory function, namely, spatial control of optical fluence and therapeutic modification of the tissue. Radiant filters may be structurally embodied using a mask (absorptive or reflective) or lenses on the anterior and/or posterior side of the pin array device (conventional, fresnel, holographic, or hybrid). For example, an absorptive or reflective mask may consist of a layer of radiant energy absorbing or reflecting dye or metal painted on either surface of the mechanical transducer. In another embodiment, a spherical surface above the pin (air-material interface) may serve as a radiant filter. Similarly, the posterior pin surface in contact with the tissue surface may also serve as a lens radiant filter. For example, a posterior pin may include a sapphire ball lens as a high thermal conductivity radiant filter. Lenses may permit angular control (focusing or divergence) of radiant energy incident on the tissue and/or allow light to be directed to a desired tissue area and/or tissue depth.

(5) Cooling System

A cooling system may provide tissue surface cooling and/or may prevent thermal damage of non-targeted, superficial layers before, during, or after the tissue contacts radiant energy. A cooling system may be applied prior to radiant emission into the tissue, during radiant emission into the tissue, and after radiant emission into the tissue. In one embodiment, the cooling system may use a convective liquid or gas forced against the targeted tissue (or pin surfaces) using a high-pressure (inlet) or low-pressure (outlet) source. An electronically controlled solenoid valve may be utilized to control the flow of liquid or gas into the control chamber and to the tissue (pin) surface thereby regulating tissue temperature profile. An example of a high-pressure inlet source may be compressed, reduced temperature, dry air. An example of a low-pressure outlet source may be a pneumatic device that may also serve as a mechanical transducer.

In a second embodiment, tissue cooling may be performed by conducting heat away from the tissue through a pinned surface. In this embodiment, it may be desirable to have a highly thermally conductive transparent material such as sapphire. In this embodiment the tip of the pin may be a highly thermally conductive material (e.g., sapphire) that is in thermal contact with a highly thermally conductive sheath or thin-wall tube surrounding the pin. Thermal conductivity of a highly thermally conductive transparent material, in some embodiments, may be over about 0.3 Wcm⁻¹K⁻¹ and/or may be over about 0.45 Wcm¹K⁻¹. A highly thermally conductive sheath or thin-walled tube surrounding a pin may be a metallic material such as aluminum. In this embodiment, a sheath or thin-walled tube and a pin may be configured and arranged to define an air gap. An air gap may permit better containment of light in a pin. Thermal energy conducted from the tissue into the tip of the pin and into the thermally conductive sheath surrounding the pin may be removed from the thin-walled tube by, for example, convective liquid or gas, a liquid-solid slurry or by an evaporative liquid.

In a third embodiment, deposition and evaporation of an evaporative liquid on the tissue surface may provide the cooling function.

When selecting temperature of the thermal fluid, duration of application of the thermal fluid and whether simultaneous radiant heating is occurring may be considered. For example in the precooling step (cooling before radiant exposure), temperature of the thermally-controlled fluid may be −50° C. if the duration of tissue contact is sufficiently short (e.g., less than 100 ms). Accordingly, thermal fluids with a higher temperature (˜10° C.) can be applied for longer times (200 ms).

During radiant exposure, temperature of the thermal fluid may be determined in part by the effective heat transfer coefficient between the tissue and fluid, and heat transfer coefficient between the thermal fluid and the pin, and rate of temperature increase due to absorption of radiant energy in the tissue. In this step, temperature of the cooling fluid may be reduced below 0° C. (e.g., −10° C.) in order to remove thermal energy from tissue regions not targeted for damage. In cases when a substantially higher heat transfer out of the tissue through the pin is desired, a thermally insulating material (polymer) may be placed between the tissue (tissue region not in contact with the pin) so that thermal fluids with very low temperatures (−50° C.) may be used over long periods of time (e.g., up to about 30 seconds).

The operating principle in selecting temperature of the thermally-controlled fluid in the pre-cooling and radiant exposure steps may be, according to some embodiment, which the temperature reduction during pre-cooling and subsequent radiant exposure in tissue regions not targeted for damage does not result in either nonspecific cryo- or thermal-injury.

The temperature of a thermally-controlled fluid may be below about −150° C., between about −150° C. and 40° C., between about −150° C. and 0° C., between about 0° C. and 40° C., between about 27° C. and 47° C., between about 28° C. and 43° C., or over 40° C.

(6) Feedback System Between Tissue and Radiant Source

Measurement of optical properties may provide a feedback signal to the cooling system and the radiant source for delivering the radiant energy dosage to the targeted tissue. The feedback system may control the radiant energy dosimetry including pulse duration, energy, and start time of exposure. The feedback system may utilize optical or mechanical sensors. An optical feedback system may utilize the radiant source used for treatment or an alternative radiant source. Similarly an optical feedback system may utilize the radiant filters used for treatment or radiant filters specialized for the feedback system.

A mechanical feedback system may utilize a pressure gauge in a hypo- or hyperbaric pressure source. In addition a mechanical feedback system may incorporate specialized pressure, stress, or strain sensors dedicated to the feedback system. Alternatively, the feedback system may utilize ultrasonic or electrical sensors.

A feedback system may control, for example, temperature and/or flow of a thermally-controlled fluid. For example, a feedback sensor may be configured to sense tissue heating and signal a controller or control to intensify cooling and/or reduce the intensity of radiation.

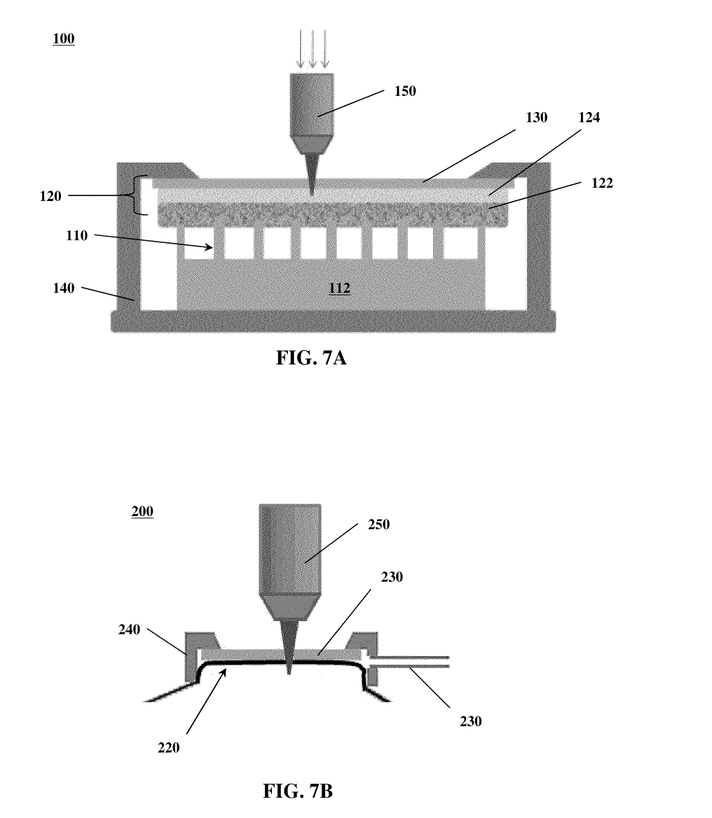

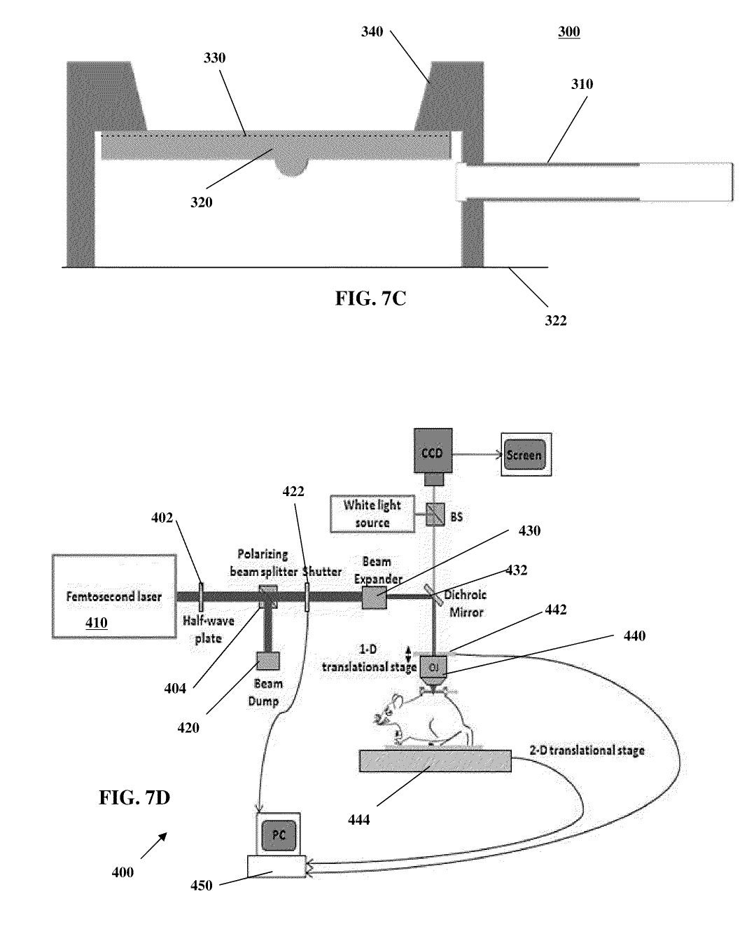

An observation system 400 may be employed, such as the one showed in FIG. 7D. A half-wave plate 402 combined with a polarizing beam splitter 404 may be positioned at the exit of the laser cavity 410 to control output pulse energy. Horizontally polarized femtosecond light 412 may be reflected by the splitter 404 and entered into a beam dump 420. Vertically polarized femtosecond light may be transmitted and used for subsurface ablation experiments in rodent skin, as indicated further below. The spatial FWHM of the femtosecond beam was reduced from 8 mm to 4 mm after passing through a beam reducer 430 (2×). The femtosecond beam was reflected by a high-pass dichroic mirror 432 with a cutoff at 700 nm and entered an objective lens 440. Visible light transmitted by the dichroic mirror 432 may be used for tissue imaging. Collimated femtosecond laser light was focused into the tissue using a 40× objective lens 440 (NA=0.55). The lens 440 was mounted on a 1-D translational stage 442 to control the axial position of the beam focus relative to skin surface. The tissue sample with the TOCD was mounted on a 2-D translational stage 444. Both translation stages 442 and 444 were controlled by a personal computer 450 equipped with Labview software. The 2-D stage 444 was programmed to move in a raster scanning mode during laser irradiation so that a continuous pattern of subsurface ablation was obtained. Fast axis of the scan moved at a speed of 2 mm/s over a distance of 4 mm with successive pulses displaced by 2 μm. Slow axis of the scan moved in steps of 20 μm over a distance of up to 6 mm. Assuming a tissue refractive index of 1.4, the beam focus was fixed at a selected depth (70 to 700 μm) below the skin surface during the raster scan. The compression may be applied for either 120 seconds for the first and third TOCDs or 300 seconds for the second TOCD before femtosecond laser ablation may be performed, allowing the intracellular water to displace from the compressed skin specimen.

(7) Tissue to be Treated

Tissue chromophores may be targeted elements in the tissue which absorb the radiant energy. Absorption of radiant energy may induce tissue therapeutic modification through photothermal, photoacoustic, or photochemical processes. The chromophores may be native tissue structures such as water, melanin or hemoglobin (blood), lipids (adipose), or synthetic such as a photodynamic dye.

(7) Tissue Optical Clearing Device

Alternative mechanical transducers and radiant source configurations are shown in FIGS. 7A and 7B. The first Tissue Optical Clearing Device (TOCD) 100 allowed usage of direct mechanical force to enhance light penetration and subsurface femtosecond photodisruption. The second TOCD 200 included a clearing effect of vacuum related compression.

As shown in FIG. 7A, the first embodiment of the TOCD 100 comprises a monolithic array of pins 110 disposed on a circular resin base 112, as further explained below. A skin sample 120 may be attached to pins 110, where the skin includes an adipose layer 122 that faces towards pins 110 and an epidermis and dermis layer 124 that faces a glass piece 130. The microscopic glass slide 130 may be attached to the epidermis layer of skin sample. A radiant source 150 delivers optical energy through the glass piece 130 to the skin sample 120. At least one C-shaped clamp 140 applies pressure to the skin sample 120 for direct mechanical forces. The scale of mechanical force may be controlled by screws on clamps or other similar tightening control devices. In one embodiment, pins 110 on the base include a diameter of at least about 2 mm, a height of at least about 2 mm, and a center-to-center distance of at least about 4 mm. The compressed tissue samples 120 were located above the pins and uncompressed tissues are between pins.

As shown in FIG. 7B, the second embodiment of the TOCD 200 comprises an optical window 230, a circumscribing brim 240, and a hollow tube 210 connected to the inside of the brim 230. In one embodiment, the optical window 210 may include a thickness, between about 0.1 mm and 10 mm. The optical window 230 may include a diameter, between about 10 mm and 100 mm. The circumscribing brim 240 may include a height of between about 0.1 mm and 10 mm. The TOCD brim 240 provided airtight seal when it was attached to the skin sample 220. After the vacuum was applied through the hollow tube 210, the skin was lifted against the inner surface of the optical window 230, causing stretching and compression of the skin tissue 220. A radiant source 250 provides optical energy to the skin sample 220.

As shown in FIG. 7C, the third embodiment of the TOCD 300 includes a planar surface of a cylindrical lens 320 was placed against the inner surface of the circular optical window 330. In one embodiment, the cylindrical lens 320 may include a length between about 1 and 100 mm, a thickness between about 0.1 and 5 mm, and a diameter between about 0.1 and 20 mm. A thin film of water is inserted into the gap between the planar surface of the cylindrical lens 320 and the optical window 330 to minimize the refractive index difference and provide a weak bond through surface tension. After a vacuum pressure was applied through a vacuum tube 310, the skin 322 surrounding the cylindrical lens was lifted against the inner surface of the optical window, causing stretching of the skin under the lens. Compared to the second TOCD, intracellular water is relatively easier and faster to displace out of the skin from under the glass lens.

Operation

A hypobaric pressure (e.g., up to about 750 mm Hg) may be applied through a small vacuum hose connected to the back side of the device.

Once the device is applied to the skin surface and the vacuum is established, skin may be positioned against the pins and compressed under individual pins. As the skin molds around the pins, tissue may become stretched and melanin concentration underneath each pin may be reduced.

Due to the mechanical pressure under each pin, tissue water may be mechanically displaced by diffusive transport with a coefficient of 1 to 1.5 mm²/s. Due to the lower pressure surrounding the pins, tissue volume may be increased and displaced water collects in the tissue surrounding the pins.

After positioning of the tissue against the pins, a first cooling step may be performed to reduce the temperature in the tissue below the pins and adjacent to the pins. The reduced temperature distribution in the tissue may be controlled by the time duration of applied cooling.

Tissue may be exposed to radiant energy during or following application of a device of the disclosure. For example, while contacting the tissue, radiant energy may be delivered through one or more pins or between pins. Cooling may be performed during radiant exposure for the purpose of removal of heat from the tissue or through the pin.

Alternatively, in some embodiments, tissue may be exposed to light after release of the vacuum and the device has been removed, but before the site has resumed its original state. Generally, methods and devices of the disclosure may be used in any tissue. For example, methods and devices, according to some embodiments, may be used with any tissue or organ covered by dermis or epidermis. In some embodiments, tissue may be visualized through a layer of mucosa.

Eight different example devices include the specifications listed in Table 4.

TABLE 4
Example device specifications

| | Device Number | | | | | | | |
|---|---|---|---|---|---|---|---|---|
| | 1 | 2 | 3 | 4 | 5 | 6 | 7 | 8 |
| Pin | 1 | 1 | 1 | 1 | 0.5 | 0.5 | 0.5 | 0.5 |
| Diameter | | | | | | | | |
| [mm] | | | | | | | | |
| Packing | 20 | 20 | 40 | 40 | 20 | 20 | 40 | 40 |
| Density | | | | | | | | |
| [%] | | | | | | | | |
| Pin | F | C | F | C | F | C | F | C |
| Surface: | | | | | | | | |
| Flat/Curved | | | | | | | | |

Similarly, blood flow may be diverted as blood is squeezed out of blood vessels underneath individual pins. Due to the applied vacuum, blood vessel diameter may be increased in the tissue surrounding the pins. This causes pooling and collection of blood in tissue between pins.

Net result of this procedure may be the creation of localized (fractional) optical tissue clearing. In contrast to optical skin clearing with hyper-osmotic chemicals this process may be rapid, occurring, for example, in a matter of a few seconds. An additional advantage may be a controlled re-distribution of water, blood and melanin, which may be specific or non-specific targeted tissue chromophores for many laser based therapeutic applications such as non-ablative skin rejuvenation, photothermal coagulation of hyper-vascular lesions, laser hair removal, adipose contouring, adipose reduction and removal, and tattoo removal. In some embodiments, methods and devices of the disclosure may be configured to be non-invasive. Such embodiments may be highly patient compliant and virtually pain free.

Localized reduction of light scattering under each pin creates channels, through which light may be delivered deeper into the tissue. This effect may be facilitated further by designing the device to include radiant filters. The anterior surface, i.e., the side facing away from the skin may feature micro-lenses as radiant filters, which help to guide and focus incoming radiation into each pin. Depending on the required light delivery depth the pin surface also may feature different optical lensing properties to allow further focusing or spreading of the light beam exiting the pin. In one embodiment, a sapphire ball lens may be used to form a high thermal conductivity radiant filter.

This allows for numerous degrees of freedom to design and tailor the device to meet specific therapeutic requirements and to optimize the therapeutic outcome based on the delivered light profile and depth. The vacuum pressure may also be varied to accommodate different skin conditions and to control the optical clearing depth.

Since the device allows fluid re-distribution (e.g., blood and water, which collect in the tissue peripheral to the pins), different optical designs may be feasible for the treatment of hyper-vascular lesions. Enlarging blood vessels allows better coagulation of vessels, which otherwise may be too small to absorb sufficient energy. Thus, different designs allow therapeutic treatment in tissue between pins as well as under pins. Micro-lens radiant filters may be placed onto different locations on the device, permitting incident laser light to be directed either into pins or into the area between pins.

The ability to deliver light deeper into tissue alleviates one of the most significant limitations in light-based therapeutic applications. Since the pin geometry results in individual light channels separated by tissue which may not be exposed to laser light, an approach similar to fractional photothermolysis may be achieved.

Tissue may be exposed to light during or following application of a device of the disclosure. For example, while contacting the tissue, light may be delivered through one or more pins or between pins. Alternatively, in some embodiments, tissue may be exposed to light after release of the vacuum and the device has been removed, but before the site has resumed its original state. Generally, methods and devices of the disclosure may be used in any tissue. For example, methods and devices, according to some embodiments, may be used with any tissue or organ covered by dermis or epidermis. In some embodiments, tissue may be visualized through a layer of mucosa.

The present disclosure includes, without limitation, a method and apparatus for modifying optical properties of biological tissue. The device described above incorporates several design features, which may improve (e.g., significantly improve) light delivery into tissues such as skin.

By reducing light scattering and re-distributing major tissue chromophores including water, hemoglobin and melanin this device may significantly improve therapeutic and diagnostic use of laser light. Devices of the disclosure may improve therapeutic applications such as laser hair removal, skin rejuvenation and photothermal coagulation of hyper-vascular lesions and non-ablative skin rejuvenation.

Potential applications include but are not limited to, laser hair removal, laser tattoo removal, photothermal coagulation of hyper-vascular lesions, and non-ablative skin rejuvenation, adipose recontouring, adipose reduction, and adipose removal.

Fractional Cooling

There may be conditions under which irradiation of tissue is correlated with adverse and/or unwanted events including, for example, undesirable tissue heating and/or photothermal damage to deeper tissue layers. Accordingly, in some embodiments of the disclosure, a system, device, and/or method of the disclosure may include measures to offset, reduce, and/or eliminate such adverse and/or unwanted events. For example, a system, device, and/or method of the disclosure may include means for fractionally cooling tissue. A fractional cooling device may include, for example, a base (e.g., a planar or substantially base) and a brim contiguous with or fixed to one side of the base, wherein the base and the brim at least partially define a flow chamber. At least a portion of the flow chamber may be defined by the surface of the tissue with which it is contacted.

A thermally-controlled composition (e.g., fluid) may flow through a closed flow chamber (e.g., a flow chamber in contact with skin), for example, via an inlet port and outlet port. The inlet and outlet ports may be positioned anywhere relative to each other. For example, in some embodiments, the inlet and outlet ports may be positioned from adjacent to each other to as far apart as possible.

In some embodiments, an optical clearing device may include a flow chamber for fractional cooling. In some embodiments, fractional cooling may include contacting at least a portion of the tissue that will be, is being, and/or was irradiated with a thermally-controlled fluid. This may include, for example, contacting a tissue with an optical clearing device having an inlet and an outlet for a thermally-controlled composition and flowing the thermally-controlled fluid through the inlet and outlet ports such that it contacts at least a portion of the tissue as shown in FIG. 4C.

Nonlimiting examples of a thermally-controlled fluid may include a gas (e.g., nitrogen, oxygen), a mixture of gases (e.g., air) or a liquid (e.g., water). In some embodiments of the disclosure, a thermally-controlled fluid may be purified (e.g., filtered, sanitized, sterilized, or otherwise treated to remove or destroy materials) before and/or after contacting tissue. For example, where the tissue may be sensitive to infection, a thermally-controlled fluid may be pre-sterilized. In another example, a thermally-controlled fluid may be post-sterilized (e.g., filter-sterilized, chemically-sterilized, and/or autoclaved) after contact with a tissue having or potentially having contagions or other biohazardous components. A thermally-controlled fluid may be processed to add or remove water (e.g., vapor) prior to contacting tissue in some embodiments. For example, it may be desirable to humidify air to reduce the potential for tissue dehydration. Alternatively, it may be desirable to dehumidify air to reduce the possibility of contacting tissue with a microbe or other unwanted material or fouling of other system or device components. A fluid may be dehumidified using water removal filters and/or desiccant driers.

The temperature of a thermally-controlled fluid may be regulated using any structures, devices, and/or methods available. For example, the temperature of a thermally-controlled fluid may be regulated using a heat pump or a Ranque-Hilsch vortex tube. Dehumidification may be desired in some embodiments to prevent heat pump or vortex malfunction. In a specific example embodiment, a thermally-controlled fluid may be water that is pre-chilled in an ice-water bath.

In some embodiments, a system or device of the disclosure may include a thermally-controlled fluid and a thermal regulator that controls the temperature of the thermally-controlled fluid. A thermal regulator may include a thermal sensor, a thermostat, and/or a refrigerant. A thermally-controlled fluid may be maintained at a constant temperature or a substantially constant temperature (e.g., ±1° C., ±2° C., ±5° C.), according to some embodiments. In other embodiments, the temperature of a thermally-controlled fluid may ramp, oscillate, or otherwise vary in a regular or irregular manner as the tissue is illuminated. Increasing the velocity and/or turbulence of a thermally-controlled fluid may escalate heat loss from tissue. Tissue heat removal may be additionally aided by minimizing the chamber volume and using internal chamber features, such as a pinned surface, which disrupt laminar flow.

In some embodiments a system or device of the disclosure may include thermal sensors configured and arranged to monitor, for example, the temperature of at least a portion of (a) a tissue at or near a site of illumination, (b) an optical clearing device, and/or (c) a thermally-controlled fluid. A thermal sensor or combination of thermal sensors may also be configured and arranged to monitor, for example, a temperature difference between at least a portion of (a) a tissue at or near a site of illumination, (b) an optical clearing device, and/or (c) a thermally-controlled fluid.

A device or system of the disclosure may include, in some embodiments, a vacuum pump in fluid communication with and located downstream of an exit valve. The action of a vacuum pump may seal the device against a tissue surface and/or actuate or enhance fluid flow. A device or system of the disclosure may include, in some embodiments, one or more valves (e.g., a solenoid valve) in fluid communication with an inlet and/or an exit port. A valve may partially or completely regulate thermally-controlled fluid influx and/or efflux from a flow chamber.

Thermal regulation may, according to some embodiments, reduce the temperature of superficial tissue layers prior to or during optical radiation in order to target photothermal change in deeper tissue layers. In addition, systems, methods, and devices may include thermal regulation for lateral spatial control of heat flux out of tissue. For example, heat flux out of irradiated tissue regions may be different from regions where laser irradiation does not enter. In some cases, cooling following radiant exposure may be performed.

According to some embodiments, a method of the disclosure may include administering an optical treatment or therapy. Non-limiting examples of optical treatment or therapy may include blood vessel coagulation, hair removal, wrinkle removal, adipose recontouring, adipose reduction, and adipose removal (fat removal), and melanoma hyperthermia.

Skin and pins may be cooled convectively and/or evaporatively due to the cold substance injected within the chamber. Pins may comprise an optically transparent high thermal conductivity material including plastic, glass, sapphire and/or diamond. Sides of pins may be coated, according to some embodiments, with an opaque high thermal conductivity material (e.g. copper). This coating may further enhance heat flux from skin to convective fluid.

As shown in FIG. 4D, a fractional cooling system may be configured and arranged to have one or more heat flux pathways. For example, heat may be transferred by conduction through skin and/or convection from a skin surface to a thermally-controlled fluid (e.g., a cold fluid) (Heat Flux Pathway A). Also, heat may be transferred by conduction through skin, conduction through a metallic pin coating, and/or convection from a metallic pin coating to a thermally-controlled fluid (e.g., a cold fluid) (Heat Flux Pathway B). Heat may be transferred by conduction through skin, conduction through a pin, conduction through a metallic pin coating, and/or convection from metallic pin coating to a thermally-controlled fluid (e.g., a cold fluid) (Heat Flux Pathway C). Components of an optical clearing device may be configured and arranged (e.g., by adjusting the pin coating and/or diameter) to strike a desired balance between these pathways. In some embodiments, the balance may include portions of all three pathways while in others one or two of these three pathways may be substantially or completely excluded.

As shown in FIG. 4E, a sapphire ball lens, according to some embodiments, may be positioned at the pin tip and a highly conductive (e.g., metal) tube or sheath is placed around the pin with an air-space between the pin and inner surface of the tube. The sapphire ball lens is thermally connected to the highly conductive tube so that thermal energy in the tissue can be conducted into the ball lens, though the highly conductive tube and dissipated with the thermally-controlled fluid.

Systems, devices, and methods according to some embodiments, may include a feedback mechanism. For example, a feedback mechanism may include activating or deactivating a radiant source when a temperature reaches a threshold value. In some embodiments, tissue may be pre-cooled and a feedback mechanism may trigger a radiant source once the tissue temperature has been lowered by a pre-set desired value (e.g., 30° C.). In some embodiments, a feedback mechanism may be configured to shut down a radiant source if the temperature of a tissue exceeds a pre-set value (e.g., 40° C.).

Without limiting any particular embodiment of the disclosure, fractional cooling may provide one or more of the following benefits: tissue alignment with radiant source, efficacy, controllability, repeatability, and uniformity of tissue temperature profile, treatment procedure safety, and environmental sensitivity. In addition, formation of the chamber may be useful in that stabilizing the tissue surface against the chamber allows alignment of the tissue perpendicular to the radiant source. In some embodiments, an optical clearing device may include a pinned chamber surface that increases the surface area of skin in contact with cold fluid and increases turbulence of the fluid stream, which may increase heat flux from the tissue surface. Controllability of the technique and device may be enhanced by fine regulation of the fluid flow rate, temperature, and flow duration in the environmentally isolated chamber. Repeatability and uniformity of temperature profile of skin may be enhanced at least in part because the chamber is isolated from exterior environmental conditions and flow is uniform across the chamber surface. Treatment procedure safety may be enhanced due to tissue alignment with radiant source and enhanced efficacy, controllability, repeatability, and uniformity of tissue temperature profile, and temperature feedback device.

A thermally-controlled fluid may include one or more gasses such as nitrogen, oxygen, and air. A gaseous thermally-controlled fluid may be cooled using a vortex tube. Although systems, devices, and/or methods of the disclosure may include materials like R-12 and R-134a in some embodiments, cooling a thermally-controlled fluid with a vortex tube, may, in some embodiments, reduce or obviate the use of these or other such materials.

Wearable Systems and Devices

In some embodiments of the disclosure, a tissue such as skin or adipose, may be treated with optical radiation from an array of light emitting diodes (LEDs). Contacting a tissue with LED radiation may effect a photo-induced change in a targeted tissue region. Conditions that may be treated LED radiation may include hair removal, wrinkle removal, and fat removal or shaping. A system, device, and method of the disclosure may be used independently or in conjunction with other treatment modalities such as ultrasound, radio-frequency (RF), or optical.

A device may be structurally embodied as an array of LEDs which may be positioned against the skin, resulting in the formation of a closed chamber between the LED array and skin, as shown in FIG. 5. LEDs may function as a source of radiant energy, radiant filters (LED bulb), and may integrate tissue optical clearing and cooling technologies into a single device.

A system, device, or method of the disclosure may be suited for integration with a mechanical tissue clearing device. Application of mechanical forces to the tissue may provide at least three important functions: first, intracellular and interstitial water may be moved out of a targeted tissue volume causing spatial distribution of scatterers to be modified and thereby reduce light scattering and water absorption; second application of mechanical forces may increase or decrease blood volume fraction and perfusion in selected tissue regions; and third application of mechanical forces may increase or decrease the tissue volume or thickness. In this embodiment, a material surface enclosing each LED or bulb may also function as a mechanical transducer or pin. An array of LEDs with bulbs may comprise a pin array in contact with the tissue. The LED bulbs may be forced against skin using negative gas pressure in the chamber, positive gas pressure in the form of an inflatable cuff placed behind the LED array, an elastomeric material, gravity, or combinations thereof.

A system, device, or method of the disclosure may be suited for integration with a tissue cooling device. Tissue cooling may reduce the temperature of superficial tissue layers prior to or during optical radiation in order to target photo-induced change in deeper tissue layers Skin may be cooled directly by spraying cryogen or chilled fluid within the chamber. Alternatively, skin may be cooled indirectly by reducing the temperature of a LED bulb array. An array of LED bulbs may be convectively cooled by spraying cryogen or chilled fluid either on the back surface of the LED array or within each LED bulb, as shown in FIG. 5. The bulb covering each LED may be constructed entirely of an optically transparent high thermal conductivity material such as sapphire. Alternatively, sidewalls of LED bulbs may be constructed of an opaque high thermal conductivity metal (e.g., copper). In a different embodiment, skin may be cooled using convectively cooled tubing placed beneath the LED bulbs. Tubing may additionally function to optically clear the skin by mechanically transducing the forces acting on the LED bulbs as illustrated by the right-most pin shown in FIG. 5.

Without limiting any particular embodiment of the disclosure, a system, device, or method of the disclosure may provide one or more of the following benefits: cost reduction, ease of patient/operator use, and treatment procedure efficacy, repeatability, and safety. Replacement of a conventional laser source with an LED array provides reduction in cost and ease of operation. Simplicity of device operation may permit use in different environments because the device may operate without sophisticated controls, operator expertise, and implementation facilities, thereby reducing operational costs. Efficacy and repeatability of the treatment procedure may be enhanced by integrating tissue clearing and tissue cooling technologies with an environmentally isolated chamber. Repeatability of skin temperature profile may be enhanced because the chamber is isolated from exterior environmental conditions. Uniformity of skin temperature may be enhanced by regular spacing of LED array elements which provide consistent subsurface heating and superficial cooling. In some applications, safety of the procedure may be enhanced by utilizing a low-power steady-state heat transfer regime in contrast to conventional high-power transient regimen of laser therapeutic techniques.

According to some embodiments of the disclosure, an optical clearing device may optionally include a tissue cooling feature and/or may be configured and arranged into a single, wearable device. A wearable system or device of the disclosure may be suitable for in-home therapy or use. In some embodiments, a wearable system or device may be used for therapeutic procedures such as hair removal, wrinkle removal, and fat removal or shaping. A system, device, and method of the disclosure may provide a non-invasive alternative to existing invasive surgical procedures.

Radio Frequency Sources

According to some embodiments of the disclosure, a system, device, and method may include contacting a tissue with a radio frequency energy. For example, a tissue clearing device may include a radio frequency source. A radio frequency source may be combined with tissue cooling and/or laser irradiation. Without limiting any particular embodiment, a tissue clearing system, device, or method that includes a radio frequency source (“an RF tissue clearing device”) may be used in connection with cellulite treatment, acne vulgaris treatment, and wrinkle removal.

Application of a mechanical force in associate with radio frequency (RF) exposure may provide one or more of the following. First, intracellular and interstitial water may be moved out of a targeted tissue volume causing spatial distribution of scattering particles to be modified and thereby reduce light scattering and reducing absorption of RF energy in these areas. Second, application of mechanical forces may increase or decrease blood volume fraction and perfusion and modulate tissue's natural response to maintain hydration. Third, application of mechanical forces may increase or decrease the tissue volume or thickness. Modifying tissue thickness may be important in application of RF energy to selected chromophores such as cellulite.

Application of RF energy in conjunction with the tissue clearing device may be integrated functionally and structurally with tissue clearing/tissue cooling methods, systems, and devices to aid RF tissue therapy. The integrated technique/device optically clears and cools superficial layers of tissue (e.g., skin) therefore protecting it from thermal damage during therapeutic RF and/or light treatments. In addition the tissue clearing/cooling device may be configured to provide dynamic cooling of RF electrodes that are integrated into the device.

The functional/structural synergism of an RF tissue treatment application is described below and illustrated in Table 5. According to some embodiments, a system or device of the disclosure may displace water (an RF chromophore) and remove heat from the superficial layers of the tissue. This may enhance an RF procedure by reducing chromophore (water) density and initial temperature of non-target (superficial) tissue thus constraining thermal damage to targeted (deeper) tissue such as reticular dermis or adipose. If a combined RF/optical treatment is desirable, optical properties of superficial tissue may be modified, potentially allowing more light (fluence) to reach target chromophores.

TABLE 5
Functional/structural synergism of an RF tissue treatment application

| Functional | | | | | |
|---|---|---|---|---|---|
| Elements | Potential Structure | | | | |
| Radiant | Laser | Radio | Microwave | X-ray | LED |
| Source | | Frequency | | | |
| Mechanical | Chamber | Pins | Vacuum | RF probe | |
| Transducer | | | | | |
| Radiant Filters | Mask (e.g. | Lenses | | | |
| | RF probe) | | | | |
| Cooling | Chamber | Pins | RF probe | Convective | Evaporative |
| | | | | Liquid | Liquid |
| Feedback of | Optical | Temperature | Mechanical | Electrical | |
| Optical | Feedback | Feedback | Feedback | Feedback | |
| Properties to | | | | | |
| Source | | | | | |
| Tissue | Chamber | Pins | Vacuum | RF probe | |
| Position and | | | | | |
| Hold | | | | | |
| Tissue and | Melanin | Hemoglobin | Water | Dye | |
| Chromophores | | | | | |

RF probes may provide many benefits to the other functional elements of the system. For example, RF probes may contribute in full or part to the radiant energy provided to the tissue. Secondly, RF probes may contribute in full or part to the mechanical transduction function of the system. Mono or bi-polar RF probes may be structurally embodied within the chamber of the device and may serve to compress or stretch tissue, locally modifying tissue chemical content (e.g., hydration) and therefore optical/thermal properties. Third, RF probes may contribute in full or part to filtering optical radiant energy. For example, RF probes placed on the base of the chamber device may block (mask) optical radiant energy from reaching tissue, and RF probes placed along the sides of pins may guide (reflect) optical radiant energy toward pin tips. Fourth, RF probes placed along the sides or near pin tips may enhance cooling functionality of the device since metal RF probes are excellent heat conductors. Fifth, RF probes may serve additional functionality as temperature or electrical resistance feedback probes utilizing the excellent heat and electrical conductance properties of metal. Finally, RF probes may serve to position and hold tissue prior to and during treatment in the same manner as pins.

In the examples, the tissue optical clearing device was applied to enhance femtosecond beam penetration and subsurface cavitations in ex vivo rat skin samples. The successful demonstration of the device showed its potential benefits to new light-based therapies for reshaping or removing adipose tissue.

### EXAMPLES

The following examples are put forth so as to provide those of ordinary skill in the art with a complete disclosure and description of how the devices, compositions, systems, and/or methods claimed herein are made and evaluated, and are intended to be purely exemplary and are not intended to limit the scope of devices, compositions, systems, and/or methods. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Tissue Optical Clearing Device (TOCD) on Rat Skin

The first TOCD embodiment 100 allowed usage of direct mechanical force to enhance light penetration and subsurface femtosecond photodisruption. The second TOCD embodiment 200 was designed to demonstrate the clearing effect of vacuum related compression.

The first embodiment of the TOCD 100 comprises a monolithic array of pins 110 disposed on a circular resin base 112. A sample may be attached to pins with the adipose layer 122 facing towards pins. A piece of microscopic glass slide was attached to the epidermis layer of skin sample. At least one C-shaped clamp applies pressure to the skin sample for direct mechanical forces. The scale of mechanical force was controlled by screws on clamps. Pins on the base had a diameter of 2 mm, a height of 2 mm, and a center-to-center distance of 4 mm. The compressed tissues were located above the pins and uncompressed tissues are between pins.

The second embodiment of the TOCD 200 comprises a circular optical window, a circumscribing brim, and a hollow tube connected to the inside of the brim. In one embodiment, the optical window may include a thickness, between about 0.1 mm and 10 mm. The optical window may include a diameter, between about 10 mm and 100 mm. The circumscribing brim may include a height of between about 0.1 mm and 10 mm. The TOCD brim provided airtight seal when it was attached to the skin on the rat. After the vacuum was applied through the hollow tube, the skin was lifted against the inner surface of the optical window, causing stretching and compression of the skin tissue.

Tissue Specimens

Dead mature rats were obtained from Functional Optical Imaging Laboratory at the University of Texas at Austin. They were either preserved in freezer until the experiments or scheduled for experiments immediately after death. Hairs of the rats were shaved off and further removed using chemical hair removal agent before conducting any experiments. The skin including the epidermis to the adipose layer was sliced off for the first TOCD embodiment 100. The skin was left on the rat body for the second TOCD 200 embodiment.

White Light Photographic Observations

The TOCD 100 embodiment was applied for 1 min to the ex vivo rat skin. Transmission and back-reflection white light photographic images of the epidermal surface were recorded during application of the device using a Canon A530 digital camera (Tokyo, Japan).

Subsurface Femtosecond Photodisruption

All photodisruption experiments were performed using a femtosecond laser (Coherent Hidra 10, Santa Clara, Calif.) operating at a central wavelength of 800 nm and FWHM of 140 fs. The laser delivered femtosecond pulses of pulse energy up to 0.9 mJ at a tunable repetition rate of 1 to 1000 Hz. Collimated femtosecond laser beam was focused into the tissue using a 40× objective lens (NA=0.55). The lens was mounted on a 1-D translation stage to control the vertical position of the beam focus relative to skin surface. The tissue sample with the TOCD was mounted on a 2-D translation stage controlled by PC. The translation stage was programmed to move in a raster scanning mode during the irradiation so that continuous photodisruption was obtained. The beam focus was fixed at a certain depth below the epidermal surface to produce subsurface femtosecond photodisruption. Immediately after laser irradiation, the samples were fixed in Formalin solution and sent to commercial biology lab for making histology slices. The slices were stained with haematoxylin and eosin dyes and examined with a white light microscope.

Results

White Light Photographic Observations

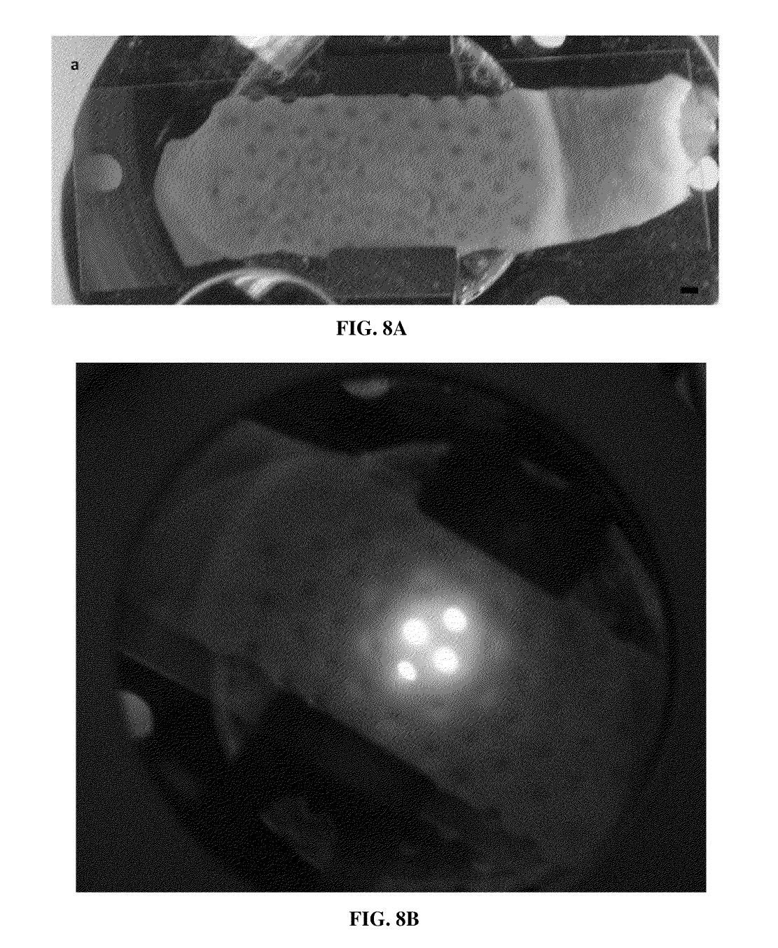

Application of the TOCD 100 embodiment to ex vivo rat skin produced mechanical forces to the skin above the pins. Normal skin appeared light pink in the back-reflection image is shown in FIG. 8A. Compressed skin regions appeared darker, an evidence of reduced backscattering and enhanced transmission. In transmission image, light transmission through compressed region increased dramatically, compared to the uncompressed skin nearby, as shown in FIG. 8B. Transmission enhancement due to application of the first TOCD appeared as bright spots surrounded by the uncompressed tissue regions.

Subsurface Femtosecond Ablation

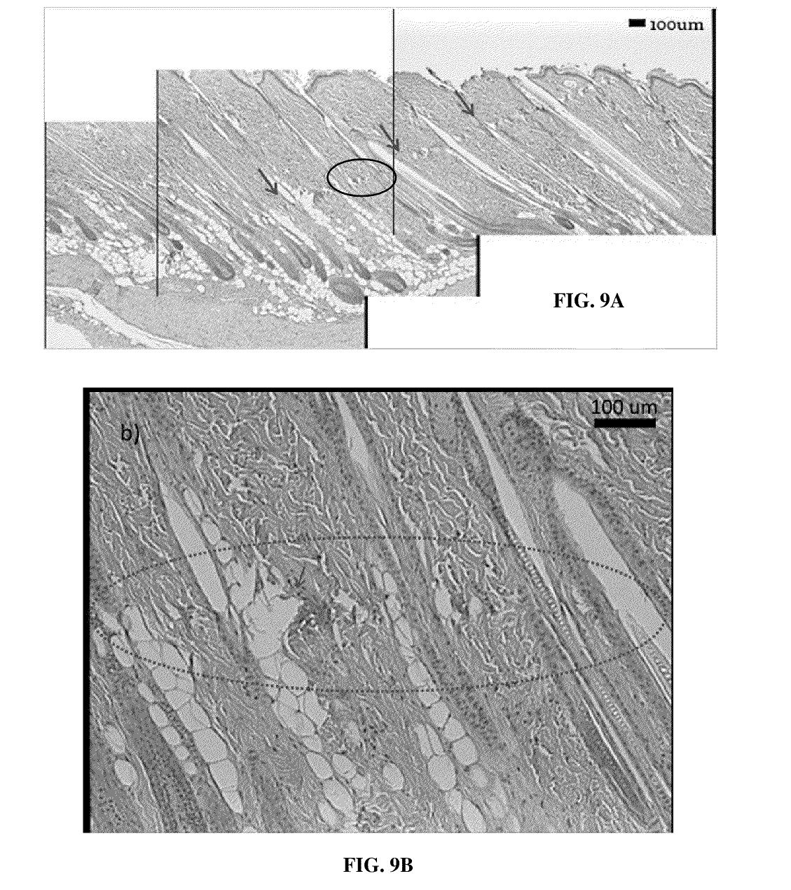

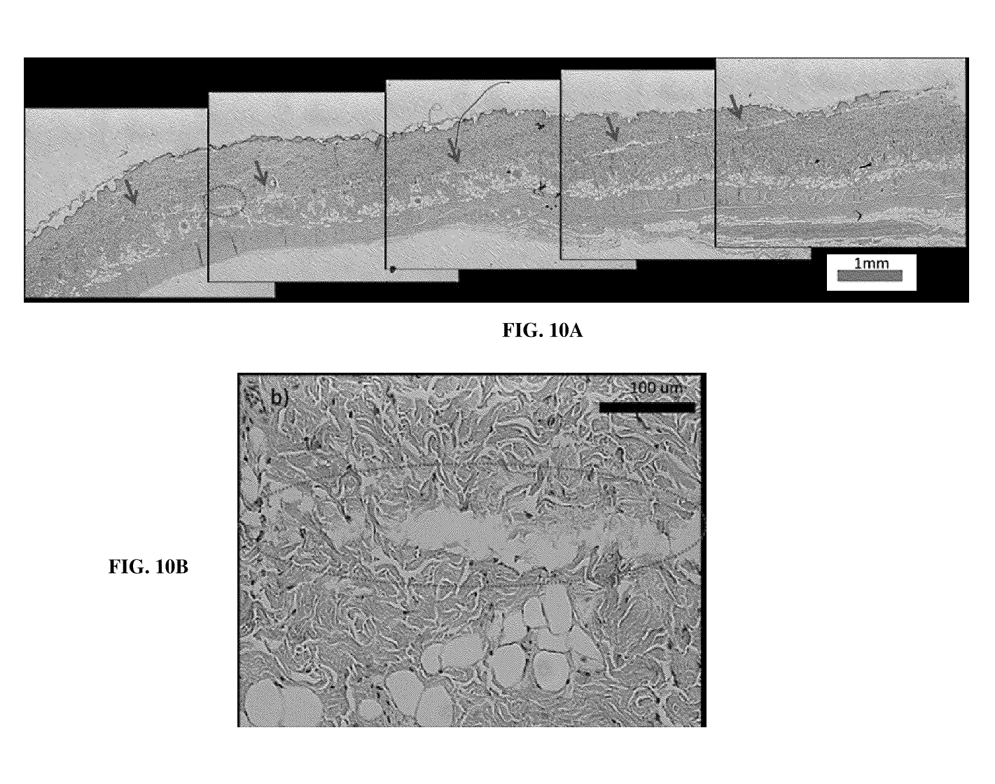

An image mosaic of histological sections of subsurface femtosecond ablation using the first TOCD 100 is shown in FIG. 9A, which indicates the region modified with the femtosecond laser (Red arrows in the figure). Subsurface cavities were observed at a depth from tens of microns to 900 μm. Average subsurface cavity diameter was approximately 30 μm at an incident pulse energy of 26 μJ measured after the objective lens. A close-up image of the circled area in FIG. 9A is displayed in FIG. 9B and shows the ablation sites within the dotted ellipse. An image mosaic of histological sections of subsurface femtosecond ablation using the second TOCD (FIG. 10A) indicates the region modified with the femtosecond laser (Red arrows in the figure). Subsurface cavities were observed at depths from tens of microns to 1.1 mm. Cavity diameter varied from 20 to 90 μm at incident pulse energy of 40 μJ. A close-up image of the circled area is displayed (FIG. 10B). Ablation cavities were observed within the dotted ellipse.

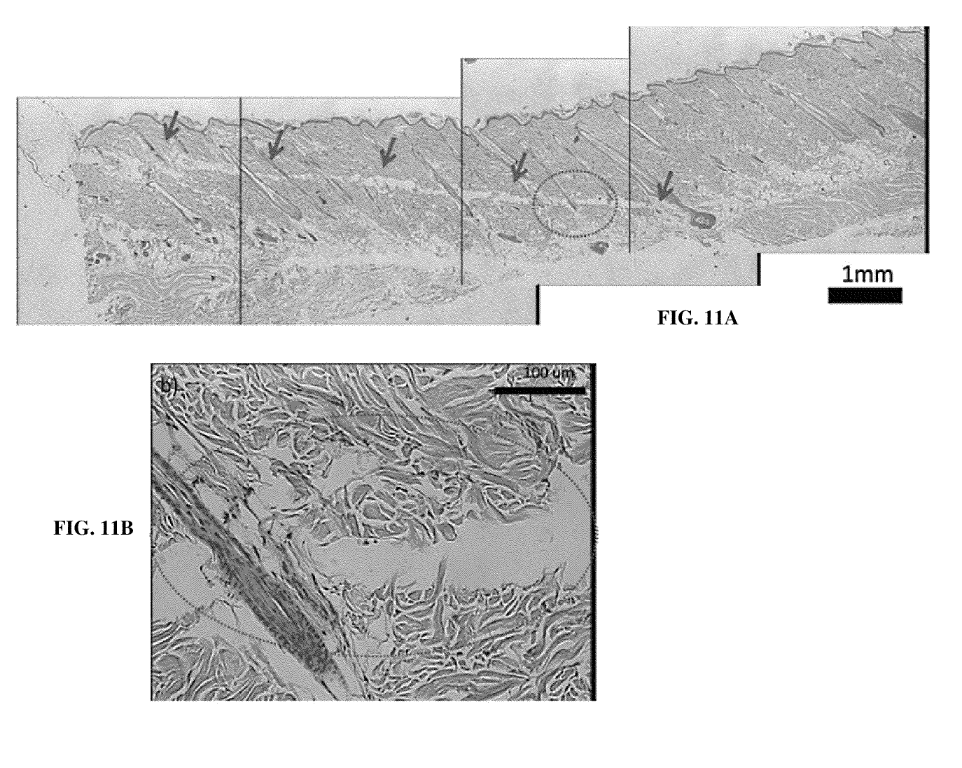

An image mosaic of histological sections of subsurface femtosecond ablation using the third TOCD (FIG. 11A) indicates the tissue region modified with the femtosecond laser pulses (Red arrows in the figure). Subsurface cavities were observed at depths from 0.3 to 1.7 mm. Cavity diameter was approximately from 30 to 120 μm at incident pulse energy of 52 μJ. A close-up image of circled area is displayed (FIG. 11B). Ablation cavities were observed within the dotted ellipse.

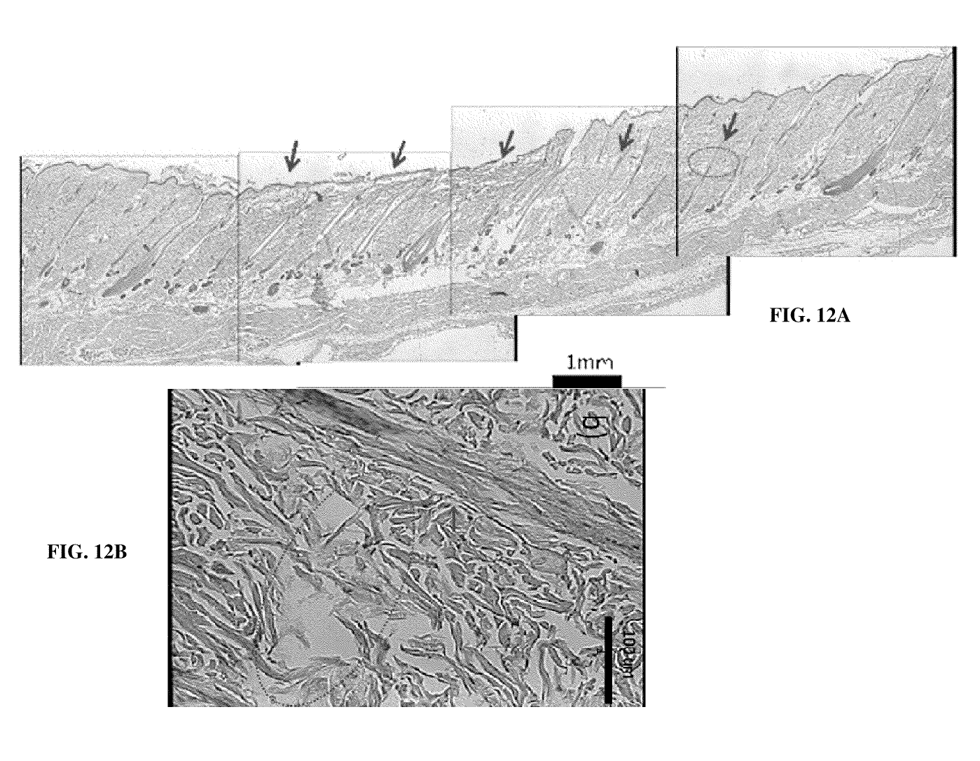

An image mosaic of histological sections of subsurface femtosecond ablation using the third TOCD (FIG. 12A) was displayed. A higher pulse energy of 69 μJ was used (52 μJ was used in FIG. 11A). Subsurface cavities were observed at depths from tens of microns to 1.1 mm. Cavity diameter was approximately from 20 to 100 μm. A close-up image of the circled area is displayed (FIG. 12B). Ablation cavities were observed within the dotted ellipse.

Discussion

The first TOCD was designed to apply mechanical compression to the dermis from the adipose side of the skin specimen. Application of the first TOCD was demonstrated to reduce dramatically scattering properties of rodent skin as recorded by reflection and transmission white light photography. Similar observations were noted by Rylander et. al. (13) Subsurface cavities were produced as deep as 900 μm below the air skin interface when the first TOCD was applied. Although the beam focus was targeted at tissue depths of 70-700 um (assuming a tissue refractive index of 1.4), subsurface ablation cavities were observed at deeper positions. The presence of deeper cavities is due in part to thinning of the tissue due to mechanical compression. Drew et. al. observed tissue thinning to 60% of initial thickness due to mechanical compression. Assuming similar compression ratios, subsurface cavities observed at 900 um, were produced at a depth of 540 um in the compressed state and within the range of targeted depths. Subsurface cavities formed a straight line pattern that sloped with respect to the air-skin interface. Although direction of histological cutting is along the slow axis of raster scanning and distance between two adjacent ablations is only 20 μm, subsurface cavities were not continuous along the line. The discontinuity is due to the relatively low pulse energy that was used. The pulse energy was about 26 μJ, less than that used for the second TOCD (40 μJ) and third TOCD (52 μJ). Mechanical force was maximal at the deep end of the cavity line so that the tissue at that location were compressed the greatest and expanded the most after the mechanical force was removed. A close-up histological image (FIG. 9B) showed that sharp edges were produced by femtosecond ablation. Both collagens (red ellipse) and adipose cells (red arrow) were ablated by femtosecond pulses. Collagens remained connected at a few locations along the ablation line. The first TOCD was applied to the adipose layer so that femtosecond light could be directly applied to the epidermis. Although the TOCD-skin geometry for the first TOCD cannot be applied clinically, the experiments allowed investigation of mechanical clearing without femtosecond light passing through the TOCD.

The second TOCD was designed to provide mechanical optical clearing of in vivo rodent skin by vacuum compression. The gauge vacuum pressure (−750 mmHg) was kept on for 300 sec before laser irradiation so that intracellular water had sufficient time to displace out of compressed regions, providing better refractive index matching and reduced scattering. Subsurface cavities were produced in rodent skin at depths as deep as 1.1 mm below the air-skin interface, 10× deeper than that produced in the intact skin. Subsurface cavities observed at 1.1 mm, were produced at a depth of 660 um in the compressed state and within the range of targeted depths. As with the second TOCD, subsurface cavities formed a straight line that sloped relative to the air-skin interface. The sloped feature resulted from a preset tilt between the laser and rodent skin sample. A higher pulse energy was used (40 μJ) and resulted in generation of larger cavities compared to the first TOCD. Higher pulse energy also increased the probability of subsurface cavity generation so that the cavities were more continuous along the sloped line feature compared to that produced by the first TOCD. Cavities were larger at the locations closer to the skin surface because less radiant energy was absorbed or scattered away by the overlying tissue structures. A close-up histological image showed that sharp edges were produced by femtosecond ablation. (FIG. 10B) Collateral damage was not observed in the image.

The third TOCD was also designed to provide mechanical optical clearing of in vivo rodent skin by vacuum compression. Addition of a cylindrical lens produced increased stretching and compression of the skin surrounding the lens and enhanced movement of intracellular water under the lens. Therefore, the third TOCD presumably provided better optical clearing than the second TOCD. The assumption was proven experimentally since subsurface cavities using the third TOCD were observed at a depth of 1.7 mm, substantially deeper than that produced by the second TOCD (1.1 mm) and 17× deeper than demonstrated previously. Assuming similar compression ratios as observed by Drew et. al., subsurface cavities observed at 1.7 mm, were produced at a depth of 1.02 mm in the compressed state and outside the range of targeted depths (70-700 um). Difference between the targeted and deepest observed depths in histological sections may be due to the cylindrical lens in the third TOCD that achieved higher compression ratios and correspondingly higher refractive indices in the compressed state. For example if the cylindrical lens achieved a compression ratio of 0.45 and tissue refractive index of 1.5, subsurface cavities could have been produced at a depth of 750 μm in the compressed state and be observed at 1.7 mm in histological images. A close-up histological image suggests that collagens were ablated precisely, whereas the hair shaft was only damaged slightly (FIG. 11B).

When the third TOCD was applied and the pulse energy was increased from 52 μJ to 69 μJ, subsurface cavities were produced at more superficial depths than those produced by low energy pulses (FIG. 12A). Cavities at depths lower than 0.5 mm did not form a straight line pattern. Increased femtosecond pulse energy caused that the power density of the beam before focus was sufficient to produce tissue ablation. Higher pulse energy also increased the size of damage area. A close-up image (FIG. 12B) of the circled area show a hair follicle damaged by the femtosecond pulses (Red arrows), a phenomenon that was not observed by low energy pulse ablation. (FIG. 12B) The hair shaft above the ablation line appeared bleached and mechanically expanded possibly due to absorption by melanin granules in the hair shaft. The observed photoeffects in the hair shaft (bleaching and mechanical expansion) were always observed (images not shown) at depths above collagen modification suggesting that melanin in the hair shaft may reduce the ablation threshold compared to that for collagen.

In summary, the tissue optical clearing devices (TOCDs) were applied directly to ex vivo and in situ rodent skin and provide optical clearing allowing deeper penetration of femtosecond laser light. In one TOCD device, femtosecond laser irradiation produced subsurface cavities in rodent skin as deep as 1.7 mm below the skin surface, 17× deeper than demonstrated previously.

The optical window of the TOCD was designed to have a flat inner surface or have a cylindrical lens attached. The cylindrical lens provided a better optical clearing effect than the flat surface. To achieve stronger vacuum compression and therefore faster optical clearing, one candidate approach is to select a different cylindrical lens or alternative bulk materials. A cylindrical lens with larger thickness may provide better compression. A glass bar may provide worse compression but its flat surface may improve focused beam transmission.

For the experiments described, wavelength dependence of the mechanical clearing effect was not examined. However, femtosecond pulses comprised of wavelengths between 1100 nm-2200 nm are scattered much less strongly than pulses with a wavelength around 775 nm. With the application of tissue optical clearing device, water concentration in the dermis is reduced so that the absorption of light with wavelengths in the 1100-2200 nm is reduced. Based on these experiments a reasonable hypothesis is that femtosecond pulses comprised of wavelengths in the 1100-2200 nm spectral range can penetrate deeper and provide superior subsurface cavities compared to 800 nm femtosecond pulses.

Optimization of the mechanical optical clearing effect may be accomplished by varying the pin geometries and femtosecond pulses comprised of various near infrared wavelengths to enhance subsurface ablation or achieve deeper penetration depths. Deeper subsurface cavity in the skin may be enhanced by modifying the inner surface of the optical window.

All references cited herein are incorporated herein by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method for controlled photodisruption in tissue using light, comprising the steps of:
a. applying a mechanical force to the tissue;
b. creating at least one localized region in the tissue; and
c. applying at least one light pulse to the at least one localized region in the tissue.

2. The method of claim 1 wherein the mechanical force is produced by at least one indenter.

3. The method of claim 2, wherein the indenter is comprised of as least one material transparent to at least one wavelength of electromagnetic radiation in the range from 100 nm-15 μm.

4. The method of claim 2, further comprising an array of indenters attached to a base pressed against said tissue.

5. The method of claim 4, further comprising a beam array consisting of at least two beamlets corresponding to at least two indenters

6. The method of claim 5, wherein at least one beamlet in the beam array is scanned in angle or position with respect to at least one indenter

7. The method of claim 1, wherein the mechanical force applied to said tissue is achieved in-part by a vacuum pressure.

8. The method of claim 1, wherein pulse of light has a full-width-half-maximum pulse duration longer than 1 fs but shorter than 1 s.

9. The method of claim 1, wherein said tissue may be imaged at least one time before or after the application of said mechanical force to the said tissue.

10. The method of claim 1, wherein application of one or more light pulses produce a targeted localized subsurface region of photodisruption in said tissue

11. The method of claim 8, wherein photodisruption is ablation

12. The method of claim 9, wherein photodisruption is plasma ablation

13. The method of claim 8, wherein photodisruption is photocoagulation

14. The method of claim 8, wherein the wavefront curvature of the incident short-pulsed light beam is varied to produce photodisruption in said localized subsurface region(s) in said tissue at a selected depth below the tissue surface.

15. The method of claim 8, wherein the incident short-pulsed light beam is scanned laterally across the tissue to produce a plurality of targeted localized subsurface regions of photodisruption in said tissue.

16. The method of claim 8, wherein localized subsurface region of photodisruption is targeted to at least one structural element in said tissue.

17. The method of claim 14, wherein structural element can include a hair follicle.

18. The method of claim 14, wherein structural element can include a fiber comprised in part of collagen.

19. The method of claim 14, wherein structural element can include a cell

20. The method of claim 14, wherein the cell is an adipocyte

21. The method of claim 14, wherein structural element can include fascia associate with cellulite

22. A device for enhancing laser-tissue interaction comprising:
a. a mechanical transducer and a pulsed radiant source to enhance light penetration and subsurface photodisruption in a sample.

23. The device of claim 22, wherein the mechanical transducer comprises an indenter.

24. The device of claim 23, further comprises a transparent back layer operable with the pulsed radiant source and the array pins, wherein the transparent back layer contacts the epidermal layer of the sample to apply pressure on the epidermal layer to enhance pulsed energy transmission into the tissue from the pulsed radiant source.

25. The device of claim 22, further comprising at least one clamp operably coupled to the array of pins to press the pins against the transparent back layer.

26. The device of claim 23, wherein the transparent back layer is substantially transparent to the pulsed radiant source energy transmission.
